(19) United States
(12) Patent Application Publication
(10) Pub. No.: US 2012/0322676 A1
(43) Pub. Date: Dec. 20, 2012

(54) COMPOSITIONS AND METHODS FOR DETECTION OF CRONOBACTER SPP. AND CRONOBACTER SPECIES AND STRAINS

(75) Inventors: Yongmei JI, Chicago, IL (US); Sheung-Mei ("Rita") Shih, Foster City, CA (US); Lovorka Degoricija, Los Gatos, CA (US); Zhaohui Zhou, San Ramon, CA (US); Craig Cummings, Pacifica, CA (US); Manohar Furtado, San Ramon, CA (US); Pius Brzoska, Woodside, CA (US); Angela Burrell, Austin, TX (US); Harrison Leong, San Francisco, CA (US); Xingwang Fang, Austin, TX (US); Mangkey Bounpheng, Austin, TX (US); Rohan Shah, Austin, TX (US)

(73) Assignee: Life Technologies Corporation, Carlsbad, CA (US)

(21) Appl. No.: 13/495,748

(22) Filed: Jun. 13, 2012

Related U.S. Application Data
(60) Provisional application No. 61/498,443, filed on Jun. 17, 2011.
(60) Provisional application No. 61/550,779, filed on Oct. 24, 2011.

(51) Int. Cl. C12Q 1/68; C40B 30/04; G01N 21/76; C07H 21/04; G01N 27/62

(52) U.S. Cl. 506/9; 435/6.12; 435/6.11; 536/23.1; 536/24.33; 536/24.32

(57) ABSTRACT

Disclosed are genomic sequences for nine strains of Cronobacter spp. (C. sakazakii—696, 701, 680; C. malonaticus—507, 681; C. turicensis—564; C. muytjensii—530; C. dublinensis—582; C. genomosp1—581) and compositions, methods, and kits for detecting, identifying and distinguishing Cronobacter spp. strains from each other and from non-Cronobacter spp. strains. Some embodiments describe isolated nucleic acid compositions unique to certain Cronobacter strains as well as compositions that are specific to all Cronobacter spp. Primer and probe compositions and methods of use of primers and probes are also provided. Kits for identification of Cronobacter spp. are also described. Some embodiments relate to computer software methods for setting a control based threshold for analysis of PCR data.

### REFERENCE TO RELATED APPLICATION

This application claims the benefit of priority of U.S. Provisional Patent Application Ser. No. 61/550,779, filed Oct. 24, 2011, and of U.S. Provisional Patent Application Ser. No. 61/498,443, filed Jun. 17, 2011, the entire contents of which applications are incorporated herein by reference in their entirety.

### EFS INCORPORATION PARAGRAPH

### Sequence Listing

The instant application contains a Sequence Listing which has been submitted in ASCII format via EFS-Web and is hereby incorporated by reference in its entirety. Said ASCII copy, created on Jun. 6, 2012, is named LT00536U.txt and is 612,204 bytes in size.

### FIELD OF THE DISCLOSURE

The present teachings relate to compositions, methods and kits for detection and identification of Cronobacter spp. and Cronobacter species and strains. More particularly, the specification describes compositions and kits comprising nucleic acid sequences specific and/or unique to Cronobacter spp. and also specific to Cronobacter species or strains, and methods of use thereof. Methods for differentially detecting Cronobacter spp. from closely related bacterial species, and the Cronobacter species from each other as well as from other bacterial species are also described.

In some embodiments, present teachings relate to computer program products including a tangible computer-readable storage medium whose contents include a program with instructions being executed on a processor so as to perform a method for PCR analysis.

### BACKGROUND

Cronobacter spp. (formerly Enterobacter sakazakii) is a bacterium within the family Enterobacteriaceae. Cronobacter are gram-negative opportunistic food-borne pathogens and are known as rare but important causes of life-threatening neonatal infections which can lead to severe diseases, such as brain abscesses, meningitis, necrotizing enterocolitis and systemic sepsis. Death has been reported in up to 40-80 percent of neonatal patients, occurring within a few hours to several days. Surviving infants may experience neurological impairment and central nervous system infection. Recently, the emergence of antibiotic-resistant strains has been observed. Effectively detecting Cronobacter in food such as contaminated powdered infant formula is extremely important from the public health and economic perspective.

The Cronobacter genus is composed of six named species, including C. sakazakii (strains—BAA-894, 680, 696, 701), C. malonaticus (strains—507, 681), C. turicensis (strains—z3032, 564), C. dublinensis (strain—582), C. muytjensii (strain—530) and C. genomosp 1 (strain—581). The C. sakazakii ST4 (sequence type as defined by Multi Locus Sequence Typing) strains (701 is an ST4 strain) are recently found to be strongly associated with neonatal meningitis (Joseph and Forsythe, 2011). So far only two complete genomes, for C. sakazakii—BAA 894 (Kucerova et al., 2010) and C. turicensis—z3032 (Stephan et al., 2011), are publicly available. Genome sequences of more species and strains are desired to study pathogenicity and evolution of the genus, as well as design molecular assays for specific detection of a species or the genus.

Design and development of molecular detection assays that differentiate or identify a target sequence that is present in organisms to be detected, and absent or divergent in organisms not to be detected is an unmet need for the definitive detection of the pathogenic Cronobacter Spp.

### SUMMARY OF THE DISCLOSURE

The present disclosure, in some embodiments, discloses the genomic sequences of nine Cronobacter strains (696, 701, 680, 507, 681, 564, 582, 530 and 581). In some embodiments, the disclosure describes isolated nucleic acid sequence compositions comprising portions of the nine Cronobacter strain genomes. In some embodiments, isolated nucleic acid sequence compositions of the disclosure comprise nucleic acid sequences unique to and/or specific to Cronobacter spp. organisms. In some embodiments, eleven strains of Cronobacter were analyzed to find sequences that are unique of specific to Cronobacter spp. organisms.

In some embodiments, isolated nucleic acid sequence compositions of the disclosure comprise nucleic acid sequences unique to and/or specific to each of the six Cronobacter species and the C. sakazakii ST4 strain. In some embodiments, isolated nucleic acid sequence compositions of the disclosure comprise nucleic acid sequences longer than 100 nucleotides unique to and/or specific to each of the six Cronobacter species and the C. sakazakii ST4 strain. In some embodiments, isolated nucleic acid sequences of the disclosure may have at least 90% sequence identity, at least 80% sequence identity, and/or at least 70% sequence identity to nucleic acid sequences comprising unique and/or specific portions of eleven strains of Cronobacter spp. organisms. In some embodiments, isolated nucleic acid sequences of the disclosure may have at least 90% sequence identity, at least 80% sequence identity, and/or at least 70% sequence identity to nucleic acid sequences comprising unique and/or specific portions of each of the six species and the C. sakazakii ST4 strain of Cronobacter spp.

In some embodiments, unique Cronobacter spp. nucleic acid sequences may comprise isolated nucleic acid molecules comprising a nucleotide sequence of —SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, fragments thereof, and/or complements thereof. In some embodiments, unique Cronobacter spp. sequences may comprise isolated nucleic acid molecules comprising a nucleotide sequence having at least a 90% sequence identity, at least 80% sequence identity and/or at least 70% sequence identity to the nucleotide sequences of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, fragments thereof and/or complements thereof.

In some embodiments, Cronobacter spp. isolated nucleic acid sequences may comprise nucleic acid molecules comprising at least 40 nucleotide sequence of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12; at least 30 nucleotide sequence of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12; at least 25 nucleotide sequence of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12; at least 20 nucleotide sequence of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12; at least 15 nucleotide sequence of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12; at least 10 nucleotide sequence of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12; any intermediate number of contiguous sequences from at least about 10 nucleotides of sequence to at least about 40 nucleotides of sequence of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, and sequences having 90% identity to the foregoing sequences.

In some embodiments, unique C. sakazakii nucleic acid sequences may comprise isolated nucleic acid molecules comprising a nucleotide sequence selected from SEQ ID NOs:16-117, fragments thereof, and/or complements thereof. In some embodiments, unique C. sakazakii sequences may comprise isolated nucleic acid molecules comprising a nucleotide sequence having at least a 90% sequence identity, at least 80% sequence identity and/or at least 70% sequence identity to the nucleotide sequences of SEQ ID NOs:16-117, fragments thereof and/or complements thereof.

In some embodiments, C. sakazakii isolated nucleic acid sequences may comprise nucleic acid molecules comprising at least a 40 nucleotide contiguous sequence of a sequence having SEQ ID NOs: 16-117; at least a 30 nucleotide contiguous sequence of a sequence having SEQ ID NOs: 16-117; at least a 25 nucleotide contiguous sequence of a sequence having SEQ ID NOs: 16-117; at least a 20 nucleotide contiguous sequence of a sequence having SEQ ID NOs: 16-117; at least a 15 nucleotide contiguous sequence of a sequence having SEQ ID NOs: 16-117; at least a 10 nucleotide contiguous sequence of a sequence having SEQ ID NOs: 16-117; any intermediate number of contiguous sequences from at least about 10 nucleotides of sequence to at least about 40 nucleotides of sequence of a sequence having SEQ ID NOs: 16-117, and sequences having 90% identity to the foregoing sequences.

In some embodiments, unique C. turicensis nucleic acid sequences may comprise isolated nucleic acid molecules comprising a nucleotide sequence having SEQ ID NOs: 118-204, fragments thereof, and/or complements thereof. In some embodiments, unique C. turicensis sequences may comprise isolated nucleic acid molecules comprising a nucleotide sequence having at least a 90% sequence identity, at least 80% sequence identity and/or at least 70% sequence identity to the nucleotide sequences of SEQ ID NOs: 118-204, fragments thereof and/or complements thereof.

In some embodiments, C. turicensis isolated nucleic acid sequences may comprise nucleic acid molecules comprising at least a 40 nucleotide contiguous sequence of a sequence having SEQ ID NOs: 118-204; at least a 30 nucleotide contiguous sequence of a sequence having SEQ ID NOs: 118-204; at least a 25 nucleotide contiguous sequence of a sequence having SEQ ID NOs: 118-204; at least a 20 nucleotide contiguous sequence of a sequence having SEQ ID NOs: 118-204; at least a 15 nucleotide contiguous sequence of a sequence having SEQ ID NOs: 118-204; at least a 10 nucleotide contiguous sequence of a sequence having SEQ ID NOs: 118-204; any intermediate number of contiguous sequences from at least about 10 nucleotides of sequence to at least about 40 nucleotides of sequence having SEQ ID NOs: 118-204, and sequences having 90% identity to the foregoing sequences.

In some embodiments, unique C. malonaticus nucleic acid sequences may comprise isolated nucleic acid molecules comprising a nucleotide sequence selected from SEQ ID NOs:205-273, fragments thereof, and/or complements thereof. In some embodiments, unique C. malonaticus sequences may comprise isolated nucleic acid molecules comprising a nucleotide sequence having at least a 90% sequence identity, at least 80% sequence identity and/or at least 70% sequence identity to the nucleotide sequences of SEQ ID NOs:205-273, fragments thereof and/or complements thereof.

In some embodiments, C. malonaticus isolated nucleic acid sequences may comprise nucleic acid molecules comprising at least a 40 nucleotide contiguous sequence of a sequence having SEQ ID NOs:205-273; at least a 30 nucleotide contiguous sequence of a sequence having SEQ ID NOs:205-273; at least a 25 nucleotide contiguous sequence of a sequence having SEQ ID NOs:205-273; at least a 20 nucleotide contiguous sequence of a sequence having SEQ ID NOs:205-273; at least a 15 nucleotide contiguous sequence of a sequence having SEQ ID NOs:205-273; at least a 10 nucleotide contiguous sequence of a sequence having SEQ ID NOs:205-273; any intermediate number of contiguous sequences having at least about 10 nucleotides to at least about 40 nucleotides of sequence of a sequence having SEQ ID NOs:205-273, and sequences having 90% identity to the foregoing sequences.

In some embodiments, unique C. muytjensii nucleic acid sequences may comprise isolated nucleic acid molecules comprising a nucleotide sequence selected from SEQ ID NOs:274-685, fragments thereof, and/or complements thereof. In some embodiments, unique C. muytjensii sequences may comprise isolated nucleic acid molecules comprising a nucleotide sequence having at least a 90% sequence identity, at least 80% sequence identity and/or at least 70% sequence identity to the nucleotide sequences of SEQ ID NOs:274-685, fragments thereof and/or complements thereof.

In some embodiments, C. muytjensii isolated nucleic acid sequences may comprise nucleic acid molecules comprising at least a 40 nucleotide contiguous sequence of a sequence having SEQ ID NOs:274-685; at least a 30 nucleotide contiguous sequence of a sequence having SEQ ID NOs:274-685; at least a 25 nucleotide contiguous sequence of a sequence having SEQ ID NOs:274-685; at least a 20 nucleotide contiguous sequence of a sequence having SEQ ID NOs:274-685; at least a 15 nucleotide contiguous sequence of a sequence having SEQ ID NOs:274-685, at least a 10 nucleotide contiguous sequence of a sequence having SEQ ID NOs:274-685; any intermediate number of contiguous sequences from at least about 10 nucleotides of sequence to at least about 40 nucleotides of sequence of a sequence having SEQ ID NOs:274-685, and sequences having 90% identity to the foregoing sequences.

In some embodiments, unique C. genomosp1 nucleic acid sequences may comprise isolated nucleic acid molecules comprising a nucleotide sequence having SEQ ID NOs:686-820, and/or complements thereof. In some embodiments, unique C. genomosp1 sequences may comprise isolated nucleic acid molecules comprising a nucleotide sequence having at least a 90% sequence identity, at least 80% sequence identity and/or at least 70% sequence identity to the nucleotide sequences of SEQ ID NOs:686-820, fragments thereof and/or complements thereof.

In some embodiments, C. genomosp1 isolated nucleic acid sequences may comprise nucleic acid molecules comprising at least a 40 nucleotide contiguous sequence of a sequence having SEQ ID NOs:686-820; at least a 30 nucleotide contiguous sequence of a sequence having SEQ ID NOs:686-820; at least a 25 nucleotide contiguous sequence of a sequence having SEQ ID NOs:686-820; at least a 20 nucleotide contiguous sequence of a sequence having SEQ ID NOs:686-820; at least a 15 nucleotide contiguous sequence of a sequence having SEQ ID NOs:686-820; at least a 10 nucleotide contiguous sequence of a sequence having SEQ ID NOs:686-820; or any intermediate number of contiguous sequences from at least about 10 nucleotides to at least about 40 nucleotides of a sequence having SEQ ID NOs:686-820, and sequences having 90% identity to the foregoing sequences

In some embodiments, unique C. dublinensis nucleic acid sequences may comprise isolated nucleic acid molecules comprising a nucleotide sequence of SEQ ID NOs: 821-1213, fragments thereof, and/or complements thereof. In some embodiments, unique C. dublinensis sequences may comprise isolated nucleic acid molecules comprising a nucleotide sequence having at least a 90% sequence identity, at least 80% sequence identity and/or at least 70% sequence identity to the nucleotide sequences of SEQ ID NOs: 821-1213, fragments thereof and/or complements thereof.

In some embodiments, C. dublinensis isolated nucleic acid sequences may comprise nucleic acid molecules comprising at least a 40 nucleotide contiguous sequence of a sequence having SEQ ID NOs: 821-1213; at least a 30 nucleotide contiguous sequence of a sequence having SEQ ID NOs: 821-1213; at least a 25 nucleotide contiguous sequence of a sequence having SEQ ID NOs: 821-1213; at least a 20 nucleotide contiguous sequence of a sequence having SEQ ID NOs: 821-1213; at least a 15 nucleotide contiguous sequence of a sequence having SEQ ID NOs: 821-1213; at least a 10 nucleotide contiguous sequence of a sequence having SEQ ID NOs: 821-1213; or any intermediate number of contiguous sequences from at least about 10 nucleotides of sequence to at least about 40 nucleotides of sequence of a sequence having SEQ ID NOs: 821-1213, and sequences having 90% identity to the foregoing sequences.

In some embodiments, unique C. sakazakii ST4 strain nucleic acid sequences may comprise isolated nucleic acid molecules comprising a nucleotide sequence having SEQ ID NOs: 1213-1278, fragments thereof, and/or complements thereof. In some embodiments, unique C. sakazakii ST4 strain sequences may comprise isolated nucleic acid molecules comprising a nucleotide sequence having at least a 90% sequence identity, at least 80% sequence identity and/or at least 70% sequence identity to the nucleotide sequences of SEQ ID NOs: 1213-1278, fragments thereof and/or complements thereof.

In some embodiments, C. sakazakii ST4 strain isolated nucleic acid sequences may comprise nucleic acid molecules comprising at least a 40 nucleotide contiguous sequence of a sequence having SEQ ID NOs: 1213-1278; at least a 30 nucleotide contiguous sequence of a sequence having SEQ ID NOs: 1213-1278; at least a 25; at least a 20 nucleotide contiguous sequence of a sequence having SEQ ID NOs: 1213-1278; at least a 15 nucleotide contiguous sequence of a sequence having SEQ ID NOs: 1213-1278; at least a 10 nucleotide contiguous sequence of a sequence having SEQ ID NOs: 1213-1278; or any intermediate number of contiguous sequences from at least about 10 nucleotides to at least about 40 nucleotides of a sequence having SEQ ID NOs: 1213-1278, and sequences having 90% identity to the foregoing sequences

In some embodiments, the disclosure describes compositions of isolated nucleic acid sequences having SEQ ID NO:13, SEQ ID NO:14, SEQ ID NO:15, fragments thereof, complements thereof and isolated nucleic acid sequence comprising at least 90% nucleic acid sequence identity to the sequences set forth above.

In some embodiments, isolated nucleic acid sequence compositions of the disclosure may further comprise one or more label, such as, but not limited to, a dye, a radioactive isotope, a chemiluminescent label, a fluorescent moiety, a bioluminescent label an enzyme, and combinations thereof.

The disclosure also describes recombinant constructs comprising nucleic acid sequences unique to Cronobacter spp. as set forth in sections above. Accordingly, a recombinant construct of the disclosure may comprise a nucleotide sequence of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, fragments thereof, complements thereof as well as nucleotide sequences having at least a 90% identity, at least 80% identity and/or at least 70% identity to the nucleotide sequences described above.

In some embodiments, recombinant constructs may comprise nucleic acid sequences unique to the Cronobacter species comprising a nucleotide sequence of SEQ ID NO: 16-1278, fragments thereof (including fragments having at least 10 contiguous nucleotides thereof), complements thereof as well as nucleotide sequences having at least a 90% identity, at least 80% identity and/or at least 70% identity to the nucleotide sequences described above.

In some embodiments, a recombinant construct of the disclosure may comprise a nucleotide sequence of SEQ ID NO:13, SEQ ID NO:14, SEQ ID NO:15, complements thereof and isolated nucleic acid sequence comprising at least 90% nucleic acid sequence identity to the sequences set forth above.

The specification also discloses methods for detection of an organism of Cronobacter spp. organism from a sample, and methods to exclude the presence of an Cronobacter spp. organism in a sample, wherein the detection of at least one nucleic acid sequence that is unique to an Cronobacter spp. is indicative of the presence of an Cronobacter spp. and the absence of detection of any nucleic acid sequence unique to an Cronobacter spp. is indicative of the absence of an Cronobacter spp. in the sample. Accordingly, a method of the disclosure, in some embodiments, may comprise detecting, in a sample, a nucleic acid sequence having at least 10 to at least 25 nucleic acids of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, and/or complementary sequences thereof, wherein detection of the nucleic acid sequence indicates the presence of a Cronobacter spp. organism in the sample. Methods of detection may also comprise identification steps and may further comprise steps of sample preparation. Such embodiments are described in detail in sections below.

In some embodiments, the specification also discloses methods of identifying the species of Cronobacter spp. from a sample, wherein detection of at least one nucleic sequence that is unique to one of the six species is indicative of the presence of that particular species. In some embodiments, such a method may comprise detecting a nucleotide sequence of SEQ ID NO: 16-1278, at least 10 contiguous nucleotide fragments thereof, complements thereof to detect the presence of one or more species of Cronobacter.

For example, in an exemplary embodiment, a method may comprise detecting, in a sample, at least one nucleic acid molecule comprising a nucleotide sequence selected from SEQ ID NO: 16-117, fragments thereof, and/or complements thereof, wherein detection of at least one of these identifies the presence of C. sakazakii. In some embodiments, detecting one of these sequences as listed above is indicative of the absence of other Cronobacter species other than C. sakazakii.

In another embodiment, a method may comprise detecting, in a sample, at least one nucleic acid molecule comprising a nucleotide sequence selected from SEQ ID NOs: 118-204, fragments thereof, and/or complements thereof, wherein detection of at least one of these sequences is indicative of the presence of C. turicensis. In some embodiments, detecting one of the sequences as listed above is indicative of the absence of other Cronobacter species other than C. turicensis.

In similar embodiment, a method may comprise detecting, in a sample, at least one nucleic acid molecule comprising a nucleotide sequence selected from SEQ ID NOs:205-273, fragments thereof, and/or complements thereof, wherein detection of at least one of these sequences is indicative of the presence of C. malonaticus. In some embodiments, detection of a sequence as described above is indicative of the absence of other Cronobacter species other than C. malonaticus.

In similar embodiment, a method may comprise detecting, in a sample, at least one nucleic acid molecule comprising a nucleotide sequence selected from SEQ ID NOs:274-685, fragments thereof, and/or complements thereof, wherein detection of at least one of SEQ ID NOs:274-685 is indicative of the presence of C. muytjensii. In some embodiments, detecting one of these sequences is indicative of the absence of other Cronobacter species other than C. muytjensii.

In similar embodiment, a method may comprise detecting, in a sample, at least one nucleic acid molecules comprising a nucleotide sequence selected from SEQ ID NO: 686-820, fragments thereof, and/or complements thereof, wherein detection of at least one of the sequences of SEQ ID NO: 686-820 is indicative of the presence of C. genomosp1 in the sample. In some embodiments, detecting the sequences described above is indicative of the absence of other Cronobacter species other than C. genomosp1.

In similar embodiment, a method may comprise detecting, in a sample, at least one nucleic acid molecule comprising a nucleotide sequence selected from SEQ ID NO: 821-1213, fragments thereof, and/or complements thereof, wherein detection of at least one of the sequences of SEQ ID NO: 821-1213 is indicative of the presence of C. dublinensis. In some embodiments, detection of these sequences as described above is indicative of the absence of other Cronobacter species other than C. dublinensis.

In another embodiment, a method may comprise detecting, in a sample, at least one nucleic acid molecule comprising a nucleotide sequence selected from SEQ ID NO: 1214-1278, fragments thereof, and/or complements thereof, wherein detection of at least one of these sequences is indicative of the presence of C. sakazakii ST4 strain. In some embodiments, detection of a sequence of SEQ ID NO: 1214-1278, a fragment thereof or a complement thereof is indicative of the absence of other Cronobacter species other than C. sakazakii ST4 strain.

Some embodiments describe methods of distinguishing a Cronobacter spp. from Enterobacter strains and may comprise: detecting at least one of a nucleic acid sequence having a nucleic acid sequence of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, fragments thereof, complements thereof and/or sequences comprising at least 90% nucleic acid sequence identity thereof, wherein detection of at least one of the nucleic acid sequences identifies Cronobacter spp. In other embodiments, not detecting at least one of a nucleic acid sequence selected from nucleotides described by either SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, fragments thereof, complements thereof and/or sequences comprising at least 90% nucleic acid sequence identity thereof may be used to exclude the presence of Cronobacter spp. in a sample.

Some methods for identifying and/or detecting Cronobacter spp. in a sample may comprise using a nucleotide sequence composition of the disclosure for detection. Exemplary compositions of the disclosure used for detection methods may comprise, but are not limited to, SEQ ID NO:13, SEQ ID NO:14, SEQ ID NO:15, complements thereof, isolated nucleic acid sequence comprising at least 90% nucleic acid sequence identity to the sequences set forth above and/or labeled derivatives thereof.

Some embodiments of the present disclosure are kits for detection of Cronobacter spp. A kit of the disclosure may comprise one or more isolated nucleic acid sequences of the disclosure as set forth herein. Some nucleic acid compositions of the disclosure may comprise primers for amplification of target nucleic acid sequences from a contaminating Cronobacter spp. that may be present in a sample. Some nucleic acid compositions of the disclosure may comprise probes for the detection of target nucleic acid sequences and/or amplified target nucleic acid regions from a contaminating Cronobacter spp. present in a sample. Probes and primers comprised in kits may be labeled. Kits may additionally comprise one or more components such as, but not limited to: buffers, enzymes, nucleotides, salts, reagents to process and prepare samples, probes, primers, agents to enable detection and control nucleotides. Each component of a kit of the disclosure may be packaged individually or together in various combinations in one or more suitable container means. Kits of the disclosure, in some embodiments, may be used to distinguish the presence of non-Cronobacter type bacteria. Some embodiments are also kits for identification of the species of Cronobacter spp. present in a sample.

Some embodiments of the disclosure relate to computer software algorithms and computer software based methods for standardizing analysis of data obtained during PCR reactions (such as real-time PCR). A computer based method may comprise setting an “optimal threshold value setting” based on a pre-defined percentage of the positive control's maximum plateau value (called dRN). Algorithms and software methods of the disclosure are described in detail in sections below and may advantageously allow for uniform results despite varied user expertise levels and across different labs and test site settings.

### BRIEF DESCRIPTION OF THE DRAWINGS

Some specific example embodiments of the disclosure may be understood by referring, in part, to the following description and the accompanying drawings, wherein:

FIG. 1 is a phylogenetic analysis and shows the phylogenetic tree inferred on 100 core genes, the presence of genes from the pan-genome, and the presence of putative virulence genes. The values on the branches are bootstrap values based on 1,000 replicates. FIG. 1A is the neighbor joining tree inferred based on the concatenated DNA sequence alignment of 100 Cronobacter core genes (85,059 nt); FIG. 1B is maximum parsimony tree inferred based on the presence and absence of the 6,156 genes in the pan genome; FIG. 1C is maximum parsimony tree inferred based on the presence and absence of 174 putative virulence genes, including fimbrial clusters, iron uptake system, some C. sakazakii specific genes, and putative type VI seCretion system.

FIG. 2 is a flowchart showing a software method for PCR data analysis, in accordance with some embodiments of the disclosure.

FIG. 3A-3D shows schematic diagrams of algorithms comprising multiple example software modules that perform methods for PCR data analysis, in accordance with certain embodiments of the disclosure.

FIG. 4A shows results for PCR data analysis with artificially set “high” CBT (above the control threshold); and FIG. 4B shows results for PCR data analysis with artificially set “low” CBT (below the control threshold), in comparison to a CBT set by methods of the present disclosure.

FIG. 5 show data demonstrating that setting a threshold using CBT methods and algorithms of the present disclosure provide consistent analysis of PCR data between five different users.

FIGS. 6A and 6B depict results of a CBT threshold procedure and show steps of the CBT method where Step 1 comprising position threshold at plateau of positive control and record instrument dRn value which equals 3.32754 is shown in FIG. 6A; and Step 2 shows position threshold at a pre-defined % of plateau of positive control determined in Step 1 which is equal to 0.332754 is shown in FIG. 6B.

FIG. 7A and FIG. 7B show the amount of variation in threshold setting that would be needed to change the CT by >2.

### DETAILED DESCRIPTION OF EXEMPLARY EMBODIMENTS

For purposes of interpreting this specification, the following definitions will apply and whenever appropriate, terms used in the singular will also include the plural and vice versa. In the event that any definition set forth below conflicts with the usage of that word in any other document, including any document incorporated herein by reference, the definition set forth below shall always control for purposes of interpreting this specification and its associated claims unless a contrary meaning is clearly intended (for example in the document where the term is originally used). It is noted that, as used in this specification and the appended claims, the singular forms “a,” “an,” and “the,” include plural referents unless expressly and unequivocally limited to one referent. The use of “or” means “and/or” unless stated otherwise. For illustration purposes, but not as a limitation, “X and/or Y” can mean “X” or “Y” or “X and Y”. The use of “comprise,” “comprises,” “comprising,” “having,” “include,” “includes,” and “including” are interchangeable and open terms not intended to be limiting. Furthermore, where the description of one or more embodiments uses the term “comprising,” those skilled in the art would understand that, in some specific instances, the embodiment or embodiments can be alternatively described using the language “consisting essentially of” and/or “consisting of”. The term “and/or” means one or all of the listed elements or a combination of any two or more of the listed element.

The section headings used herein are for organizational purposes only and are not to be construed as limiting the described subject matter in any way. All literature cited in this specification, including but not limited to, patents, patent applications, articles, books, and treatises are expressly incorporated by reference in their entirety for any purpose. In the event that any of the incorporated literature contradicts any term defined herein, this specification controls. While the present teachings are described in conjunction with various embodiments, it is not intended that the present teachings be limited to such embodiments. On the contrary, the present teachings encompass various alternatives, modifications, and equivalents, as will be appreciated by those of skill in the art.

The practice of the present embodiments may employ conventional techniques and descriptions of organic chemistry, polymer technology, molecular biology (including recombinant techniques), cell biology, biochemistry, and immunology, which are within the skill of the art, in light of the present teachings. Some conventional techniques include, but may not be limited to, oligonucleotide synthesis, hybridization, extension reactions and detection of hybridization using a label. Specific illustrations of suitable techniques may be described in example herein below. However, other equivalent conventional procedures may also be used. General conventional techniques and their descriptions can be found in standard laboratory manuals such as Genome Analysis: A Laboratory Manual Series (Vols. I-IV), PCR Primer: A Laboratory Manual, and Molecular Cloning: A Laboratory Manual (all from Cold Spring Harbor Laboratory Press, 1989), Gait, “Oligonucleotide Synthesis: A Practical Approach” 1984, IRL Press, London, Nelson and Cox (2000), Lehninger, Principles of Biochemistry 3^{rd} Ed., W. H. Freeman Pub., New York, N.Y. and Berg et al. (2002) Biochemistry, 5^{th} Ed., W. H. Freeman Pub., New York, N.Y. all of which are herein incorporated in their entirety by reference for all purposes.

The terms “amplifying” and “amplification” are used in a broad sense and refer to any technique by which a target region, an amplicon, or at least part of an amplicon, is reproduced or copied (including the synthesis of a complementary strand), typically in a template-dependent manner, including a broad range of techniques for amplifying nucleic acid sequences, either linearly or exponentially. Some non-limiting examples of amplification techniques include primer extension, including the polymerase chain reaction (PCR), reverse transcription polymerase chain reaction (RT-PCR), asynchronous PCR (A-PCR), and asymmetric PCR (AM-PCR), strand displacement amplification (SDA), multiple displacement amplification (MDA), nucleic acid strand-based amplification (NASBA), rolling circle amplification (RCA), transcription-mediated amplification (TMA), and the like, including multiplex versions, and combinations thereof. Descriptions of certain amplification techniques can be found in, among other places, Molecular Cloning, A Laboratory Manual, Cold Spring Harbor Press, 3d ed., 2001 (hereinafter “Sambrook and Russell”); Sambrook et al.; Ausubel et al.; PCR Primer: A Laboratory Manual, Diffenbach, Ed., Cold Spring Harbor Press (1995); Msuih et al., J. Clin. Micro. 34:501-07 (1996); McPherson; Rapley; U.S. Pat. Nos. 6,027,998 and 6,511,810; PCT Publication Nos. WO 97/31256 and WO 01/92579; Ehrlich et al., Science 252:1643-50 (1991); Favis et al., Nature Biotechnology 18:561-64 (2000); Protocols & Applications Guide, rev. 9/04, Promega, Madison, Wis.; and Rabenau et al., Infection 28:97-102 (2000).

The terms “amplicon,” “amplification product” and “amplified sequence” are used interchangeably herein and refer to a broad range of techniques for increasing polynucleotide sequences, either linearly or exponentially and can be the product of an amplification reaction. An amplicon can be double-stranded or single-stranded, and can include the separated component strands obtained by denaturing a double-stranded amplification product. In certain embodiments, the amplicon of one amplification cycle can serve as a template in a subsequent amplification cycle. Exemplary amplification techniques include, but are not limited to, PCR or any other method employing a primer extension step. Other nonlimiting examples of amplification include, but are not limited to, ligase detection reaction (LDR) and ligase chain reaction (LCR). Amplification methods can comprise thermal-cycling or can be performed isothermally. In various embodiments, the term “amplification product” and “amplified sequence” includes products from any number of cycles of amplification reactions.

As used herein, the “polymerase chain reaction” or PCR is a an amplification of nucleic acid consisting of an initial denaturation step which separates the strands of a double stranded nucleic acid sample, followed by repetition of (i) an annealing step, which allows amplification primers to anneal specifically to positions flanking a target sequence; (ii) an extension step which extends the primers in a 5′ to 3′ direction thereby forming an amplicon polynucleotide complementary to the target sequence, and (iii) a denaturation step which causes the separation of the amplicon from the target sequence (Mullis et al., eds, The Polymerase Chain Reaction, BirkHauser, Boston, Mass. (1994). Each of the above steps may be conducted at a different temperature, preferably using an automated thermocycler (Applied Biosystems LLC, a division of Life Technologies Corporation, Foster City, Calif.). If desired, RNA samples can be converted to DNA/RNA heteroduplexes or to duplex cDNA by methods known to one of skill in the art.

As used herein, “amplifying” and “amplification” refers to a broad range of techniques for increasing polynucleotide sequences, either linearly or exponentially. Exemplary amplification techniques include, but are not limited to, PCR or any other method employing a primer extension step. Other nonlimiting examples of amplification include, but are not limited to, ligase detection reaction (LDR) and ligase chain reaction (LCR). Amplification methods may comprise thermal-cycling or may be performed isothermally. In various embodiments, the term “amplification product” includes products from any number of cycles of amplification reactions.

In certain embodiments, amplification methods comprise at least one cycle of amplification, for example, but not limited to, the sequential procedures of: hybridizing primers to primer-specific portions of target sequence or amplification products from any number of cycles of an amplification reaction; synthesizing a strand of nucleotides in a template-dependent manner using a polymerase; and denaturing the newly-formed nucleic acid duplex to separate the strands. The cycle may or may not be repeated.

Descriptions of certain amplification techniques can be found, among other places, in H. Ehrlich et al., Science, 252:1643-50 (1991), M. Innis et al., PCR Protocols: A Guide to Methods and Applications, Academic Press, New York, N.Y. (1990), R. Favis et al., Nature Biotechnology 18:561-64 (2000), and H. F. Rabenau et al., Infection 28:97-102 (2000); Sambrook and Russell, Molecular Cloning, Third Edition, Cold Spring Harbor Press (2000) (hereinafter “Sambrook and Russell”), Ausubel et al., Current Protocols in Molecular Biology (1993) including supplements through September 2005, John Wiley & Sons (hereinafter “Ausubel et al.”).

The term “label” refers to any moiety which can be attached to a molecule and: (i) provides a detectable signal; (ii) interacts with a second label to modify the detectable signal provided by the second label, e.g. FRET; (iii) stabilizes hybridization, i.e. duplex formation; or (iv) provides a capture moiety, i.e. affinity, antibody/antigen, ionic complexation. Labelling can be accomplished using any one of a large number of known techniques employing known labels, linkages, linking groups, reagents, reaction conditions, and analysis and purification methods. Labels include light-emitting compounds which generate a detectable signal by fluorescence, chemiluminescence, or bioluminescence (Kricka, L. in Nonisotopic DNA Probe Techniques (1992), Academic Press, San Diego, pp. 3-28). Another class of labels are hybridization-stabilizing moieties which serve to enhance, stabilize, or influence hybridization of duplexes, e.g. intercalators, minor-groove binders, and cross-linking functional groups (Blackburn, G. and Gait, M. Eds. “DNA and RNA structure” in Nucleic Acids in Chemistry and Biology, 2.sup.nd Edition, (1996) Oxford University Press, pp. 15-81). Yet another class of labels effect the separation or immobilization of a molecule by specific or non-specific capture, for example biotin, digoxigenin, and other haptens (Andrus, A. “Chemical methods for 5′ non-isotopic labelling of PCR probes and primers” (1995) in PCR 2: A Practical Approach, Oxford University Press, Oxford, pp. 39-54).

The terms “annealing” and “hybridization” are used interchangeably and mean the base-pairing interaction of one nucleic acid with another nucleic acid that results in formation of a duplex or other higher-ordered structure. The primary interaction is base specific, i.e. A/T and G/C, by Watson/Crick and Hoogsteen-type hydrogen bonding.

The term “end-point analysis” refers to a method where data collection occurs only when a reaction is substantially complete.

The term “real-time analysis” refers to periodic monitoring during PCR. Certain systems such as the ABI 7700 Sequence Detection System (Applied Biosystems, Foster City, Calif.) conduct monitoring during each thermal cycle at a pre-determined or user-defined point. Real-time analysis of PCR with FRET probes measures fluorescent dye signal changes from cycle-to-cycle, preferably minus any internal control signals.

As used herein, the term “Ct” represents the PCR cycle number when the signal is first recorded as statistically significant.

The term “quenching” refers to a decrease in fluorescence of a first moiety (reporter dye) caused by a second moiety (quencher) regardless of the mechanism.

A “primer,” as used herein, is an oligonucleotide that is complementary to a portion of target polynucleotide and, after hybridization to the target polynucleotide, may serve as a starting-point for an amplification reaction and the synthesis of an amplification product. Primers include, but are not limited to, spanning primers. A “primer pair” refers to two primers that can be used together for an amplification reaction. A “PCR primer” refers to a primer in a set of at least two primers that are capable of exponentially amplifying a target nucleic acid sequence in the polymerase chain reaction.

The term “probe” comprises a polynucleotide that comprises a specific portion designed to hybridize in a sequence-specific manner with a complementary region of a specific nucleic acid sequence, e.g., a target nucleic acid sequence. In certain embodiments, the specific portion of the probe may be specific for a particular sequence, or alternatively, may be degenerate, e.g., specific for a set of sequences. In certain embodiments, the probe is labeled. The probe can be an oligonucleotide that is complementary to at least a portion of an amplification product formed using two primers.

The terms “complement” and “complementary” as used herein, refer to the ability of two single stranded polynucleotides (for instance, a primer and a target polynucleotide) to base pair with each other, where an adenine on one strand of a polynucleotide will base pair to a thymine or uracil on a strand of a second polynucleotide and a cytosine on one strand of a polynucleotide will base pair to a guanine on a strand of a second polynucleotide. Two polynucleotides are complementary to each other when a nucleotide sequence in one polynucleotide can base pair with a nucleotide sequence in a second polynucleotide. For instance, 5′-ATGC and 5′-GCAT are complementary.

A “label” refers to a moiety attached (covalently or non-covalently), or capable of being attached, to an oligonucleotide, which provides or is capable of providing information about the oligonucleotide (e.g., descriptive or identifying information about the oligonucleotide) or another polynucleotide with which the labeled oligonucleotide interacts (e.g., hybridizes). Labels can be used to provide a detectable (and optionally quantifiable) signal. Labels can also be used to attach an oligonucleotide to a surface.

A “fluorophore” is a moiety that can emit light of a particular wavelength following absorbance of light of shorter wavelength. The wavelength of the light emitted by a particular fluorophore is characteristic of that fluorophore. Thus, a particular fluorophore can be detected by detecting light of an appropriate wavelength following excitation of the fluorophore with light of shorter wavelength.

The term “quencher” as used herein refers to a moiety that absorbs energy emitted from a fluorophore, or otherwise interferes with the ability of the fluorescent dye to emit light. A quencher can re-emit the energy absorbed from a fluorophore in a signal characteristic for that quencher, and thus a quencher can also act as a fluorophore (a fluorescent quencher). This phenomenon is generally known as fluorescent resonance energy transfer (FRET). Alternatively, a quencher can dissipate the energy absorbed from a fluorophore as heat (a non-fluorescent quencher).

As used herein, “detecting” or “detection” refers to the disclosure or revelation of the presence or absence in a sample of a target polynucleotide sequence or amplified target polynucleotide sequence product. The detecting can be by end point, real-time, enzymatic, and by resolving the amplification product on a gel and determining whether the expected amplification product is present, or other methods known to one of skill in the art.

The presence or absence of an amplified product can be determined or its amount measured. Detecting an amplified product can be conducted by standard methods well known in the art and used routinely. The detecting may occur, for instance, after multiple amplification cycles have been run (typically referred to an end-point analysis), or during each amplification cycle (typically referred to as real-time). Detecting an amplification product after multiple amplification cycles have been run is easily accomplished by, for instance, resolving the amplification product on a gel and determining whether the expected amplification product is present. In order to facilitate real-time detection or quantification of the amplification products, one or more of the primers and/or probes used in the amplification reaction can be labeled, and various formats are available for generating a detectable signal that indicates an amplification product is present. For example, a convenient label is typically a label that is fluorescent, which may be used in various formats including, but are not limited to, the use of donor fluorophore labels, acceptor fluorophore labels, fluorophores, quenchers, and combinations thereof. Assays using these various formats may include the use of one or more primers that are labeled (for instance, scorpions primers, amplifluor primers), one or more probes that are labeled (for instance, adjacent probes, TaqMan® probes, light-up probes, molecular beacons), or a combination thereof. The skilled person will understand that in addition to these known formats, new types of formats are routinely disclosed. The present invention is not limited by the type of method or the types of probes and/or primers used to detect an amplified product. Using appropriate labels (for example, different fluorophores) it is possible to combine (multiplex) the results of several different primer pairs (and, optionally, probes if they are present) in a single reaction. As an alternative to detection using a labeled primer and/or probe, an amplification product can be detected using a polynucleotide binding dye such as a fluorescent DNA binding dye. Examples include, for instance, SYBR® Green dye or SYBR® Gold dye (Molecular Probes). Upon interaction with the double-stranded amplification product, such polynucleotide binding dyes emit a fluorescence signal after excitation with light at a suitable wavelength. A polynucleotide binding dye such as a polynucleotide intercalating dye also can be used.

PCR is an extremely powerful technique for amplifying specific polynucleotide sequences, including genomic DNA, single-stranded cDNA, and mRNA among others. Various methods of conducting PCR amplification and primer design and construction for PCR amplification will be known to those of skill in the art. Generally, in PCR a double-stranded DNA to be amplified is denatured by heating the sample. New DNA synthesis is then primed by hybridizing primers to the target sequence in the presence of DNA polymerase and excess dNTPs. In subsequent cycles, the primers hybridize to the newly synthesized DNA to produce discreet products with the primer sequences at either end. The products accumulate exponentially with each successive round of amplification.

The DNA polymerase used in PCR is often a thermostable polymerase. This allows the enzyme to continue functioning after repeated cycles of heating necessary to denature the double-stranded DNA. Polymerases that are useful for PCR include, for example, Taq DNA polymerase, Tth DNA polymerase, Tfl DNA polymerase, Tma DNA polymerase, Tli DNA polymerase, and Pfu DNA polymerase. There are many commercially available modified forms of these enzymes including. AmpliTaq® and AmpliTaq Gold® both available from Applied Biosystems. Many are available with or without a 3- to 5′ proofreading exonuclease activity. See, for example, Vent® and Vent®. (exo-) available from New England Biolabs.

Other suitable amplification methods include the ligase chain reaction (LCR) (e.g., Wu and Wallace, Genomics 4, 560 (1989) and Landegren et al., Science 241, 1077 (1988)), transcription amplification (Kwoh et al., Proc. Natl. Acad. Sci. USA 86, 1173 (1989)), and self-sustained sequence replication (Guatelli et al., Proc. Nat. Acad. Sci. USA, 87, 1874 (1990)) and nucleic acid based sequence amplification (NABSA). (See, U.S. Pat. Nos. 5,409,818, 5,554517, and 6,063,603). The latter two amplification methods include isothermal reactions based on isothermal transcription, which produce both single-stranded RNA (ssRNA) and double-stranded DNA (dsDNA) as the amplification products in a ratio of about 30 or 100 to 1, respectively.

As used herein, the term “analyzing” refers to evaluating and comparing the results of a method. In some exemplary embodiments, “analyzing” refers to evaluating and comparing the results of a sample tested to a second sample and/or to a control in a method of the disclosure.

As used herein, “complement” and “complements” are used interchangeably and refer to the ability of a nucleotide, a polynucleotide or two single stranded polynucleotides (for instance, a primer and a target polynucleotide) to base pair with each other, where an adenine on one strand of a polynucleotide will base pair to a thymine or uracil on a strand of a second polynucleotide and a cytosine on one strand of a polynucleotide will base pair to a guanine on a strand of a second polynucleotide. Two polynucleotides are complementary to each other when a nucleotide sequence in one polynucleotide can base pair with a nucleotide sequence in a second polynucleotide. For instance, 5′-ATGC-3′ and 5′-GCAT-3′ are complementary.

As used herein the term “complementary nucleotide sequence” and “complementary sequences” refers to a (second) nucleotide sequence which, by base pairing, is the complement of a first nucleotide sequence. For example, a forward strand with the sequence 5′-ATGGC-3′ would have the complementary nucleotide sequence 3′-TACCG-5′, also termed the “reverse strand.”

As used herein, the term “contacting” as used herein refers to the hybridization between a primer and its substantially complementary region. “Contacting” may also refer to bringing in contact at least two moieties (reagents, cells, nucleic acids) to bring about a change or a reaction in one or all the moieties. The process of contacting may also comprise “incubating” (contacting for a certain time lengths) and/or incubating at certain temperatures to bring about the change or reaction.

As used herein, “DNA” refers to deoxyribonucleic acid in its various forms as understood in the art, such as genomic DNA, cDNA, isolated nucleic acid molecules, vector DNA, and chromosomal DNA. “Nucleic acid” refers to DNA or RNA in any form. Examples of isolated nucleic acid molecules include, but are not limited to, recombinant DNA molecules contained in a vector, recombinant DNA molecules maintained in a heterologous host cell, partially or substantially purified nucleic acid molecules, and synthetic DNA molecules. Typically, an “isolated” nucleic acid is free of sequences which naturally flank the nucleic acid (i.e., sequences located at the 5′ and 3′ ends) in the native nucleic acid or genomic DNA of the organism from which the nucleic acid is derived. Moreover, an “isolated” nucleic acid molecule, such as a cDNA molecule, is generally substantially free of other cellular material when isolated from a cell and/or culture medium when produced by recombinant techniques, and/or substantially free of chemical precursors or other chemicals when chemically synthesized.

The terms “detecting” and “detection” are used in a broad sense herein and encompass any technique by which one can determine the absence or presence of something, and/or identify a nucleic acid sequence and/or a protein encoded by a nucleic acid sequence. In some embodiments, detecting comprises quantitating a detectable signal from the nucleic acid, including without limitation, a real-time detection method, such as quantitative PCR (“Q-PCR”). In some embodiments, detecting comprises determining the sequence of a sequencing product or a family of sequencing products generated using an amplification product as the template; in some embodiments, such detecting comprises obtaining the sequence of a family of sequencing products.

As used here, “distinguishing” and “distinguishable” are used interchangeably and refer to differentiating between at least two results from substantially similar or identical reactions, including but not limited to, two different amplification products, two different melting temperatures, two different melt curves, and the like. The results can be from a single reaction, two reactions conducted in parallel, two reactions conducted independently, i.e., separate days, operators, laboratories, and so on.

As used herein, the term “Cronobacter spp.-specific nucleotide sequence” and “a nucleic acid sequence unique to Cronobacter spp.” refers broadly to nucleotide sequences specific and/or unique to the eleven strains of Cronobacter spp. and not known or found in other Enterobacter strains or in other related and/or unrelated microorganisms. These sequences are shared by all eleven Cronobacter genomes with at least 95% identity, but are at least 20% divergent in all the other 45 Enterobacter genomes. These sequences do not include sequences with at least 80% identity over 50 or more nucleotides with the GenBank bacterial, viral, fungal and plant sequences when compared using BLASTN. These include, but are not limited to, nucleic acid sequences comprised in SEQ ID NO: 1, SEQ ID NO:2, SEQ ID NO: 3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, as well as fragments, complements, and sequences having at least 90% sequence identity thereof.

As used herein, the term “homology” refers to a degree of complementarity at the nucleic acid level that can be determined by known methods, e.g. computer-assisted sequence comparisons (Basic local alignment search tool, S. F. Altschul et al., J. Mol. Biol. 215 (1990), 403 410). The term “homology” known to the skilled person describes the degree to which two or more nucleic acid molecules are related, this being determined by the concordance between the sequences. The percentage of “homology” is obtained from the percentage of identical regions in two or more sequences, taking into account gaps or other sequence peculiarities. The homology of nucleic acid molecules which are related to one another can be determined with the aid of known methods. As a rule, special computer programs with algorithms which take account of the particular requirements are employed. There can be partial homology or complete homology (i.e., identity). A partially complementary sequence that at least partially inhibits a completely complementary sequence from hybridizing to a target nucleic acid is referred to using the functional term “substantially homologous.”

The term “selectively hybridize” and variations thereof means that under appropriate stringency conditions, a given sequence (for example, but not limited to, a primer) anneals with a second sequence comprising a complementary string of nucleotides (for example but not limited to a target flanking sequence or a primer-binding site of an amplicon), but does not anneal to undesired sequences, such as non-target nucleic acids or other primers. Typically, as the reaction temperature increases toward the melting temperature of a particular double-stranded sequence, the relative amount of selective hybridization generally increases and mis-priming generally decreases. In this specification, a statement that one sequence hybridizes or selectively hybridizes with another sequence encompasses situations where the entirety of both of the sequences hybridize to one another and situations where only a portion of one or both of the sequences hybridizes to the entire other sequence or to a portion of the other sequence.

The terms “identity”, “nucleic acid sequence identity” and “sequence identity” are used interchangeably and refer to the percentage of pair-wise identical residues—following homology alignment of a sequence of a polynucleotide with a sequence in question—with respect to the number of residues in the longer of these two sequences. The term “identity” as known in the art refers to a relationship between the sequences of two or more polypeptide molecules or two or more nucleic acid molecules, as determined by comparing the sequences. In the art, “identity” also means the degree of sequence relatedness between nucleic acid molecules or polypeptides, as the case may be, as determined by the match between strings of two or more nucleotide or two or more amino acid sequences. “Identity” measures the percent of identical matches between the smaller of two or more sequences with gap alignments (if any) addressed by a particular mathematical model or computer program (i.e., “algorithms”).

The term “percent (%) nucleic acid sequence identity” with respect to a nucleic acid sequence refers to the percentage of nucleotides in a first sequence that are identical with the nucleotides in a second nucleic acid sequence of interest, after aligning the sequences and introducing gaps, if necessary, to achieve the maximum percent sequence identity. Alignment for purposes of determining percent nucleic acid sequence identity can be achieved in various ways that are known to one of skill in the art, for instance, using publicly available computer software such as NCBI-BLAST, WU-BLAST, MUMmer or MAUVE software.

Percent nucleic acid sequence identity may also be determined using the sequence comparison program NCBI-BLAST or WU-BLAST (Altschul et al., Nucleic Acids Res. 25:3389-3402 (1997)). The NCBI-BLAST sequence comparison program may be downloaded from http://www.ncbi.nlm.nih.gov or otherwise obtained from the National Institute of Health, Bethesda, Md. The WU-BLAST sequence comparison program may be downloaded from http://blast.wustl.edu/. NCBI-BLAST uses several search parameters, wherein all of those search parameters are set to default values including, for example, unmask=yes, strand=all, expected occurrences=10, minimum low complexity length=15/5, multi-pass e-value=0.01, constant for multi-pass=25, dropoff for final gapped alignment=25 and scoring matrix=BLOSUM62.

In situations where NCBI-BLAST or WU-BLAST is employed for sequence comparisons, the % nucleic acid sequence identity of a given nucleic acid sequence C to, with, or against a given nucleic acid sequence D (which can alternatively be phrased as a given nucleic acid sequence C that has or comprises a certain % nucleic acid sequence identity to, with, or against a given nucleic acid sequence D) is calculated as follows: 100 times the fraction W/Z where W is the number of nucleotides scored as identical matches by the sequence alignment program NCBI-BLAST or WU-BLAST in that program's alignment of C and D, and where Z is the total number of nucleotides in D. It will be appreciated that where the length of nucleic acid sequence C is not equal to the length of nucleic acid sequence D, the % nucleic acid sequence identity of C to D will not equal the % nucleic acid sequence identity of D to C.

As used herein, the terms “polynucleotide”, “oligonucleotide”, and “nucleic acid sequences” are used interchangeably and refer to single-stranded and double-stranded polymers of nucleotide monomers, including without limitation 2′-deoxyribonucleotides (DNA) and ribonucleotides (RNA) linked by internucleotide phosphodiester bond linkages, or internucleotide analogs, and associated counter ions, e.g., H⁺, NH₄⁺, trialkylammonium, Mg²⁺, Na⁺, and the like. A polynucleotide may be composed entirely of deoxyribonucleotides, entirely of ribonucleotides, or chimeric mixtures thereof and can include nucleotide analogs. The nucleotide monomer units may comprise any nucleotide or nucleotide analog. Polynucleotides typically range in size from a few monomeric units, e.g. 5-40 when they are sometimes referred to in the art as oligonucleotides, to several thousands of monomeric nucleotide units. Unless denoted otherwise, whenever a polynucleotide sequence is represented, it will be understood that the nucleotides are in 5′ to 3′ order from left to right and that “A” denotes deoxyadenosine, “C” denotes deoxycytosine, “G” denotes deoxyguanosine, “T” denotes thymidine, and “U” denotes deoxyuridine, unless otherwise noted.

As used herein, the terms “target polynucleotide,” “nucleic acid target” and “target nucleic acid” are used interchangeably and refer to a particular nucleic acid sequence of interest. The “target” can be a polynucleotide sequence that is sought to be amplified and can exist in the presence of other nucleic acid molecules or within a larger nucleic acid molecule. The target polynucleotide can be obtained from any source, and can comprise any number of different compositional components. For example, the target can be a nucleic acid (e.g. DNA or RNA). It will be appreciated that target polynucleotides can be cut or sheared prior to analysis, including the use of such procedures as mechanical force, sonication, restriction endonuclease cleavage, or other methods known in the art.

As used herein “preparing” or “preparing a sample” or “processing” or processing a sample” refers to one or more of the following steps to achieve extraction and separation of a nucleic acid from a sample: (1) bacterial enrichment, (2) separation of bacterial cells from the sample, (3) cell lysis, and (4) nucleic acid extraction and/or purification (e.g., DNA extraction, total DNA extraction, genomic DNA extraction, RNA extraction). Embodiments of the nucleic acid extracted include, but are not limited to, DNA, RNA, mRNA and miRNA.

As used herein, “presence” refers to the existence (and therefore to the detection) of a reaction, a product of a method or a process (including but not limited to, an amplification product resulting from an amplification reaction), or to the “presence” and “detection” of an organism such as a pathogenic organism or a particular strain or species of an organism.

The term “primer” refers to a polynucleotide and analogs thereof that are capable of selectively hybridizing to a target nucleic acid or a “template,” a target region flanking sequence or to a corresponding primer-binding site of an amplification product; and allows detection of a double-stranded nucleic acid formed by hybridization or the synthesis of a sequence complementary to the corresponding polynucleotide template, flanking sequence or amplification product from the primer's 3′ end. Typically a primer can be between about 10 to 100 nucleotides in length and can provide a point of initiation for template-directed synthesis of a polynucleotide complementary to the template, which can take place, in the presence of appropriate enzyme(s), cofactors, substrates such as nucleotides and the like.

As used herein, the term “amplification primer” refers to an oligonucleotide, capable of annealing to an RNA or DNA region adjacent a target nucleic acid sequence, and serving as an initiation primer for nucleic acid synthesis under suitable conditions well known in the art. Typically, a PCR reaction employs a pair of amplification primers including an “upstream” or “forward” primer and a “downstream” or “reverse” primer, which delimit a region of the RNA or DNA to be amplified.

As used herein, the term “primer-binding site” refers to a region of a polynucleotide sequence, typically a sequence flanking a target region and/or an amplicon that can serve directly, or by virtue of its complement, as the template upon which a primer can anneal for any suitable primer extension reaction known in the art, for example, but not limited to, PCR. It will be appreciated by those of skill in the art that when two primer-binding sites are present on a single polynucleotide, the orientation of the two primer-binding sites is generally different. For example, one primer of a primer pair is complementary to and can hybridize with the first primer-binding site, while the corresponding primer of the primer pair is designed to hybridize with the complement of the second primer-binding site. Stated another way, in some embodiments the first primer-binding site can be in a sense orientation, and the second primer-binding site can be in an antisense orientation. A primer-binding site of an amplicon may, but need not comprise the same sequence as or at least some of the sequence of the target flanking sequence or its complement.

The terms “reporter probe” and “probe” are used interchangeably and refer to a detectable sequence of nucleotides or a detectable sequence of nucleotide analogs operable to specifically anneal with a corresponding amplicon, such as but not limited to, a target nucleic acid sequence and/or a PCR product and is further operable to be detected or identified. Reporter probes or probes may be detectable by a variety of methods, including but not limited to, detecting color, detecting radiation, fluorescence, luminescence, emitted wavelengths. In some embodiments, detecting a change in intensity, a change in radiation, a change in an emitted wavelength, a change in fluorescence, a change in luminescence, or a change in color or intensity of color may be used to identify and/or quantify a corresponding amplicon or a target polynucleotide. In one exemplary embodiment, by indirectly detecting an amplicon from a sample or processed sample, one can determine that a microorganism having a corresponding target sequence is present in a sample. Most reporter probes can be categorized based on their mode of action, for example but not limited to: nuclease probes, including without limitation TaqMan® probes; extension probes including without limitation scorpion primers, Lux™ primers, Amplifluors, and the like; and hybridization probes including without limitation molecular beacons, Eclipse probes, light-up probes, pairs of singly-labeled reporter probes, hybridization probe pairs, and the like. In certain embodiments, reporter probes may comprise an amide bond, an LNA, a universal base, and/or combinations thereof, and may include stem-loop and/or stem-less reporter probe configurations. Certain reporter probes may be singly-labeled, while other reporter probes are doubly-labeled. Dual probe systems that comprise FRET between adjacently hybridized probes are within the intended scope of the term reporter probe. In certain embodiments, a reporter probe may comprise a fluorescent reporter group and a quencher (including without limitation dark quenchers and fluorescent quenchers). Some non-limiting examples of reporter probes include TaqMan® probes; Scorpion probes (also referred to as scorpion primers); Lux™ primers; FRET primers; Eclipse probes; molecular beacons, including but not limited to FRET-based molecular beacons, multicolor molecular beacons, aptamer beacons, PNA beacons, and antibody beacons; labeled PNA clamps, labeled PNA openers, labeled LNA probes, and probes comprising nanocrystals, metallic nanoparticles and similar hybrid probes (see, e.g., Dubertret et al., Nature Biotech., 19:365-70, 2001; Zelphati et al., BioTechniques 28:304-15, 2000). In certain embodiments, reporter probes may further comprise minor groove binders including but not limited to TaqMan® MGB probes and TaqMan® MGB-NFQ probes (both from Applied Biosystems). In certain embodiments, reporter probe detection may comprise fluorescence polarization detection (see, e.g., Simeonov and Nikiforov, Nucl. Acids Res. 30:E91, 2002).

Those skilled in the art understand that as a target nucleic acid region (target sequence) is amplified by an amplification means, the complement of the primer-binding site is synthesized in the complementary amplicon or the complementary strand of the amplicon. Accordingly, it is to be understood that the complement of a primer-binding site is expressly included within the intended meaning of the term primer-binding site, as used herein.

As used herein, the term “genome” refers to the complete nucleic acid sequence, containing the entire genetic information, of a bacterium, a virus, a plasmid, a gamete, an individual, a population, a species, or a strain of a species.

As used herein, the term “pseudochromosome” refers to the concatenation, in their most likely order, of all available sequence contigs and scaffolds derived from sequencing of a bacterial genome, in which undefined gaps between contigs and scaffolds are represented by unidentified nucleobases.

As used herein, the term “genomic DNA” refers to the chromosomal DNA sequence of a gene or segment of a gene including the DNA sequence of non-coding as well as coding regions. Genomic DNA also refers to DNA isolated directly from cells, chromosomes or plasmid(s) within the genome of an organism, or cloned copies of all or part of such DNA.

As used herein the term “sample” refers to a starting material suspected of harboring a particular microorganism or group of microorganisms. A “contaminated sample” refers to a sample harboring a pathogenic microbe thereby comprising nucleic acid material from the pathogenic microbe. Examples of samples include, but are not limited to, food samples (including but not limited to samples from food intended for human or animal consumption such as processed foods, raw food material, produce (e.g., fruit and vegetables), legumes, meats (from livestock animals and/or game animals), fish, sea food, nuts, beverages, drinks, fermentation broths, and/or a selectively enriched food matrix comprising any of the above listed foods), infant formulas, infant food, water samples, environmental samples (e.g., soil samples, dirt samples, garbage samples, sewage samples, industrial effluent samples, air samples, or water samples from a variety of water bodies such as lakes, rivers, ponds etc.,), air samples (from the environment or from a room or a building), forensic samples, agricultural samples, pharmaceutical samples, biopharmaceutical samples, samples from food processing and manufacturing surfaces, and/or biological samples. A “biological sample” refers to a sample obtained from eukaryotic or prokaryotic sources. Examples of eukaryotic sources include mammals, such as a human, a cow, a pig, a chicken, a turkey, a livestock animal, a fish, a crab, a crustacean, a rabbit, a game animal, and/or a member of the family Muridae (a murine animal such as rat or mouse). A biological sample may include blood, urine, feces, or other materials from a human or a livestock animal. A biological sample can be, for instance, in the form of a single cell, in the form of a tissue, or in the form of a fluid.

A sample may be tested directly, or may be prepared or processed in some manner prior to testing. For example, a sample may be processed to enrich any contaminating microbe and may be further processed to separate and/or lyse microbial cells contained therein. Lysed microbial cells from a sample may be additionally processed or prepares to separate, isolate and/or extract genetic material from the microbe for analysis to detect and/or identify the contaminating microbe. Analysis of a sample may include one or more molecular methods. For example, according to some exemplary embodiments of the present disclosure, a sample may be subject to nucleic acid amplification (for example by PCR) using appropriate oligonucleotide primers that are specific to one or more microbe nucleic acid sequences that the sample is suspected of being contaminated with. Amplification products may then be further subject to testing with specific probes (or reporter probes) to allow detection of microbial nucleic acid sequences that have been amplified from the sample. In some embodiments, if a microbial nucleic acid sequence is amplified from a sample, further analysis may be performed on the amplification product to further identify, quantify and analyze the detected microbe (determine parameters such as but not limited to the microbial strain, pathogenecity, quantity etc.).

Recitation of numerical ranges by endpoints in this specification include all numbers subsumed within that range (e.g., 1 to 5 includes 1, 1.5, 2, 2.75, 3, 3.80, 4, 5, etc.).

Various embodiments of the present teachings relate to compositions, methods and kits for identification of a Cronobacter spp. microorganism. Cronobacter spp. is known to cause human disease, especially in infants, and hence is a pathogen that is a potential food contaminant, an environmental contaminant and may cause life threatening neo-natal infections.

One embodiment of the disclosure identifies signature sequences that are present only in Cronobacter spp. and absent in non-Cronobacter species as well as other closely related bacterial species. In some embodiments, the disclosure identifies signature sequences that are present only in a particular Cronobacter species but are absent from other Cronobacter species. These sequences can be used to design molecular assays, such as but not limited to PCR, real-time PCR assays, hybridization based methods, which specifically detect and distinguish Cronobacter spp. from non-Cronobacter species and in some embodiments specifically detect and distinguish one Cronobacter species from another. Examples of such assays are described as methods of the disclosure. In some embodiments, the assays described herein can be used for pathogen testing for the presence of Cronobacter contamination in food samples, such as but not limited to infant formula, infant and baby food products and beverages.

The signature sequences described here can be used to design compositions comprising probes and primers that can be used in one or more molecular methods of detecting the presence of a Cronobacter contaminant in a sample as well as for distinguishing different species of Cronobacter from each other (for example to identify a contaminant and/or to diagnose what organism is causing a disease while testing a clinical sample as well as for applications such as for tracking source of infection and/or identifying cause of infection. Compositions designed herein based on one or more signature sequences can be formulated and packaged into kits. Alternatively, signature sequences and fragments/complements thereof can be used in applications such as generic identification of pathogens including species of pathogens in chip arrays and/or barcodes on sequencing platforms.

The present disclosure, in some embodiments discloses nucleotide sequences specific to Cronobacter spp. (shared by all eleven strains) and discloses detection assays designed using nucleotide sequences specific for different serotypes. The specific and unique sequences (also referred to herein as signature sequences) were discovered by whole-genome sequencing of nine strains of Cronobacter spp. The entire genome sequences of the nine strains of Cronobacter spp. were sequenced and the genomic information was analyzed to design highly specific Cronobacter spp. assays. Embodiments relating to sequencing Cronobacter spp. are described in the section entitled Examples.

Various embodiments of the present teachings relate to compositions based on newly discovered genomic sequence regions specific and unique to Cronobacter spp. The unique and specific sequences of Cronobacter spp as well as sequences unique to different species of Cronobacter are described in SEQ ID NOs:1-12 and SEQ ID NOs: 16-1278. Example compositions of the disclosure include isolated sequences that are uniquely found in all the eleven strains of Cronobacter spp. but not in other closely related Enterobacter strains. These include, in some exemplary embodiments, at least isolated nucleic acid sequences described herein as SEQ ID NO: 1, SEQ ID NO: 2, SEQ ID NO: 3, SEQ ID NO: 4, SEQ ID NO:5, SEQ ID NO: 6, SEQ ID NO: 7, SEQ ID NO: 8, SEQ ID NO: 9, SEQ ID NO: 10, SEQ ID NO:11 and SEQ ID NO: 12, fragments thereof and complements thereof. Compositions of the disclosure also include sequences that are complements of, fragments of, and/or sequences comprising at least 90% nucleic acid sequence identity to the sequences of SEQ ID NO: 1, SEQ ID NO: 2, SEQ ID NO: 3, SEQ ID NO: 4, SEQ ID NO:5, SEQ ID NO: 6, SEQ ID NO: 7, SEQ ID NO: 8, SEQ ID NO: 9, SEQ ID NO: 10, SEQ ID NO:11 and SEQ ID NO: 12.

In some embodiments, isolated nucleic acid sequences of the disclosure may comprise nucleic acid molecules comprising at least a 40 nucleotide sequence SEQ ID NO: 1, SEQ ID NO: 2, SEQ ID NO: 3, SEQ ID NO: 4, SEQ ID NO:5, SEQ ID NO: 6, SEQ ID NO: 7, SEQ ID NO: 8, SEQ ID NO: 9, SEQ ID NO: 10, SEQ ID NO:11, SEQ ID NO: 12; at least a 30 nucleotide sequence of SEQ ID NO: 1, SEQ ID NO: 2, SEQ ID NO: 3, SEQ ID NO: 4, SEQ ID NO:5, SEQ ID NO: 6, SEQ ID NO: 7, SEQ ID NO: 8, SEQ ID NO: 9, SEQ ID NO: 10, SEQ ID NO:11, SEQ ID NO: 12; at least a 25 nucleotide sequence of SEQ ID NO: 1, SEQ ID NO: 2, SEQ ID NO: 3, SEQ ID NO: 4, SEQ ID NO:5, SEQ ID NO: 6, SEQ ID NO: 7, SEQ ID NO: 8, SEQ ID NO: 9, SEQ ID NO: 10, SEQ ID NO:11, SEQ ID NO: 12; at least a 20 nucleotide sequence of SEQ ID NO: 1, SEQ ID NO: 2, SEQ ID NO: 3, SEQ ID NO: 4, SEQ ID NO:5, SEQ ID NO: 6, SEQ ID NO: 7, SEQ ID NO: 8, SEQ ID NO: 9, SEQ ID NO: 10, SEQ ID NO:11, SEQ ID NO: 12; at least a 15 nucleotide sequence of SEQ ID NO: 1, SEQ ID NO: 2, SEQ ID NO: 3, SEQ ID NO: 4, SEQ ID NO:5, SEQ ID NO: 6, SEQ ID NO: 7, SEQ ID NO: 8, SEQ ID NO: 9, SEQ ID NO: 10, SEQ ID NO:11, SEQ ID NO: 12; at least a 10 nucleotide sequence of SEQ ID NO: 1, SEQ ID NO: 2, SEQ ID NO: 3, SEQ ID NO: 4, SEQ ID NO:5, SEQ ID NO: 6, SEQ ID NO: 7, SEQ ID NO: 8, SEQ ID NO: 9, SEQ ID NO: 10, SEQ ID NO:11, SEQ ID NO: 12; any intermediate number of contiguous sequences from at least about 10 nucleotides of sequence to at least about 25 nucleotides of sequence of SEQ ID NO: 1, SEQ ID NO: 2, SEQ ID NO: 3, SEQ ID NO: 4, SEQ ID NO:5, SEQ ID NO: 6, SEQ ID NO: 7, SEQ ID NO: 8, SEQ ID NO: 9, SEQ ID NO: 10, SEQ ID NO:11, SEQ ID NO: 12 and sequences having 90% identity to the foregoing sequences.

Additional example compositions of the disclosure include isolated sequences described in SEQ ID NO:16-1278 that are uniquely found in a species of Cronobacter and not in another species of Cronobacter. These sequences may be described as species-specific target nucleic acid sequences. For example, SEQ ID NOs: 16-117 are found uniquely in C. sakazakii, SEQ ID NOs:118-204 are found uniquely in C. turicensis, SEQ ID NOs:205-273 are found uniquely in C. malonaticus, SEQ ID NOs:274-685 are found uniquely in C. muytjensii, SEQ ID NOs:686-820 are found uniquely in C. dublinensis, SEQ ID NOs:821-1213 are found uniquely in C. genomosp. 1 and SEQ ID NOs:1214-1278 are found uniquely in C. sakazakii ST4 strain, respectively. These include, in some exemplary embodiments, at least isolated nucleic acid sequences as listed in SEQ ID NO:16-1278, fragments thereof and complements thereof. Compositions of the disclosure also include sequences that are complements of, fragments of, and/or sequences comprising at least 90% nucleic acid sequence identity to the sequences set forth in SEQ ID NOs:16-1278.

In some embodiments, isolated nucleic acid sequences of the disclosure may comprise nucleic acid molecules comprising at least a 40 contiguous nucleotide sequence of SEQ ID NOs:16-1278; at least a 30 contiguous nucleotide sequence of SEQ ID NOs:16-1278; at least a 25 contiguous nucleotide sequence of SEQ ID NOs:16-1278; at least a 20 contiguous nucleotide sequence of SEQ ID NOs:16-1278; at least a 15 contiguous nucleotide sequence of SEQ ID NOs:16-1278; at least a 10 contiguous nucleotide sequence of SEQ ID NOs:16-1278; and/or any intermediate number of contiguous nucleotide sequences from at least about 10 nucleotides to at least about 25 nucleotides of a sequence of SEQ ID NOs:16-1278 and/or sequences having 90% identity to the foregoing sequences.

The present disclosure also provides in some embodiments compositions comprising primer and/or probe sequences that may be used for detection, identification, quantitation and/or differential detection of a Cronobacter spp. organism. Probes and/or primers generally comprise, but are not limited to, oligonucleotide sequence having from about 10 to about 40 nucleotides. Probe and primer sequences of the disclosure are probes and primers designed to hybridize to a signature sequence, such as a Cronobacter specific signature sequence such as SEQ ID NOs: 1-12 and such as to a Cronobacter species specific signature sequence such as SEQ ID NOs: 16-1278. Exemplary probe and/or primer compositions of the disclosure include, but are not limited to, an isolated nucleic acid molecules having nucleic acid sequences comprised in SEQ ID NO: 13, SEQ ID NO: 14, SEQ ID NO: 15, and/or nucleic acid sequences having at least 90% sequence identity to nucleic acid sequences comprised in SEQ ID NO: 13, SEQ ID NO: 14, SEQ ID NO: 15.

In some embodiments, exemplary probe and/or primer sequences set forth above comprising or derived from SEQ ID NO: 13, SEQ ID NO: 14, SEQ ID NO: 15 may also comprise a label or may be a derivative thereof. A label may include, but is not limited to, a dye, a radioactive isotope, a fluorescent label, a bioluminescent label, a chemiluminescent label, an enzyme. A dye in some embodiments may be a fluorescein dye, a rhodamine dye, a cyanine dye, such as but not limited to FAM™ dye, and/or a VIC® dye.

In some embodiments, probes and/or primers of the disclosure for detection, identification, quantitation and/or differential detection methods and/or steps that are described in sections below. These methods may comprise embodiments such as hybridization that utilize one or more probe sequences of the disclosure, such as, but not limited, to sequences comprising or derived from SEQ ID NO: 13, SEQ ID NO: 14, SEQ ID NO: 15; embodiments such as amplification (e.g., PCR) utilizing at least one primer pair of the disclosure, such as, but not limited, to sequences comprising or derived from SEQ ID NO: 13, SEQ ID NO: 14, SEQ ID NO: 15; embodiments such as multiplex amplification using multiple primer pairs, such as, but not limited, to sequences comprising or derived from SEQ ID NO: 13, SEQ ID NO: 14, SEQ ID NO: 15; embodiments such as quantitative detection (e.g., by real-time PCR) of amplified DNA using at least one probe and at least one primer pair.

Embodiments of the disclosure also relate to designing additional probe and/or primer sequences based on unique regions specific to and shared by the eleven strains of Cronobacter spp. described herein. Several programs and algorithms may be used to design primers and/or probes based on the nucleotide sequences specific to Cronobacter spp. that are disclosed in the present specification. Probe or primer compositions of the disclosure may be designed and synthesized by methods known in the art in light of the teachings of the present disclosure and the sequences described herein. In some embodiments, a probe or a primer may comprise a sequence having as few as 10 nucleic acids, at least 15, at least 20 and at least about 25 nucleotides in length to at least about 40 nucleotides in length may be used.

Recombinant constructs comprising a sequence of the disclosure, including for example a signature sequence, a probe and/or a primer sequence of the disclosure.

Some embodiments describe methods for detection and identification of one or more unique sequences in a target nucleic acid extracted from or present in a sample suspected of containing an Enterobacter to identify the microorganism as Cronobacter spp. Cronobacter spp. specific and unique sequences may be identified alone or in any combination in order to identify or determine the presence of Cronobacter spp. Exemplary sequences that are unique to Cronobacter spp. are described herein as SEQ ID NO: 1, SEQ ID NO: 2, SEQ ID NO: 3, SEQ ID NO: 4, SEQ ID NO:5, SEQ ID NO: 6, SEQ ID NO: 7, SEQ ID NO: 8, SEQ ID NO: 9, SEQ ID NO: 10, SEQ ID NO:11 and SEQ ID NO: 12.

Methods of the disclosure may be used for diagnostic detection and testing methods (such as for food safety testing, infant formula safety testing, baby food testing, diagnostic patient testing in people or animals that are infected with Cronobacter) and are useful to prevent and protect against Cronobacter spp. based human/animal infections.

In some embodiments, methods for detection of Cronobacter spp. may comprise detecting in a sample at least one (or more) of a nucleic acid sequence selected from the group consisting of SEQ ID NO: 1, SEQ ID NO: 2, SEQ ID NO: 3, SEQ ID NO: 4, SEQ ID NO:5, SEQ ID NO: 6, SEQ ID NO: 7, SEQ ID NO: 8, SEQ ID NO: 9, SEQ ID NO: 10, SEQ ID NO:11, SEQ ID NO: 12, fragments thereof, and complements thereof, wherein detection of one of the at least one nucleic acid sequences identifies Cronobacter spp. Methods may also employ sequences that have at least 90% nucleic acid sequence identity to these sequences.

An exemplary testing method may comprise: preparing a sample which may comprise: a) processing a sample to extract any genetic material contained in the sample and to render the genetic material amenable to detection steps (e.g., isolating nucleic acid from a sample); b) providing a composition of the disclosure comprising at least one isolated nucleotide sequence of an Cronobacter spp.-specific nucleotide sequence (such as but not limited to at least one nucleic acid sequence having the sequence of SEQ ID NO: 1, SEQ ID NO: 2, SEQ ID NO: 3, SEQ ID NO: 4, SEQ ID NO:5, SEQ ID NO: 6, SEQ ID NO: 7, SEQ ID NO: 8, SEQ ID NO: 9, SEQ ID NO: 10, SEQ ID NO:11, SEQ ID NO: 12, a fragment of the foregoing nucleic acids (also referred to as fragments thereof), a nucleic acid having from at least 10 to at least 25 nucleotides of contiguous sequences of the foregoing sequences, complements thereof and/or sequences comprising at least 90% nucleic acid sequence identity thereof); c) contacting at least one Cronobacter spp.-specific isolated nucleotide sequence with the sample (processed sample); and d) detecting hybridization of the at least one Cronobacter spp.-specific nucleotide sequence to a complementary nucleotide sequence in the sample. Detecting one or more nucleotide sequences that are unique to Cronobacter spp. are indicative that the test sample contains Cronobacter spp. Embodiments of the disclosure also describe quantitative assays by which one of skill in the art, in light of this disclosure, may quantify the amount of Cronobacter spp. in the sample.

detecting the species of Cronobacter comprising:

A method of the disclosure may comprise detecting a species-specific target nucleic acid sequence comprising detecting the presence of at least one nucleic acid selected from SEQ ID NOs:16-1278, wherein the detection of a nucleic acid having SEQ ID NO: 16-117 is indicative of the presence of C. sakazakii, the detection of a nucleic acid having SEQ ID NOs:118-204 is indicative of the presence of C. turicensis, the detection of a nucleic acid having SEQ ID NOs:205-273 is indicative of the presence of C. malonaticus; the detection of a nucleic acid having SEQ ID NOs:274-685 is indicative of the presence of C. muytjensii, the detection of a nucleic acid having SEQ ID NOs:686-820 is indicative of the presence of C. dublinensis; the detection of a nucleic acid having SEQ ID NOs:821-1213 is indicative of the presence of C. genomosp. 1; and the detection of a nucleic acid having SEQ ID NOs:1214-1278 is indicative of the presence of C. sakazakii ST4 strain.

In some embodiments, a nucleic acid may be isolated from a sample prior to practicing a method of the disclosure by isolating nucleic acids by methods known in the art to isolate nucleic acids from samples. Samples of various kinds as described in sections above may be amenable to the methods. In some embodiments, methods of the disclosure may comprise testing a food sample for contamination by Cronobacter spp. and may comprise isolating nucleic acid from a food sample having a selectively enriched food matrix.

Detecting the at least one nucleic acid sequence from a sample may be performed by one or more technologies, such as, but not limited to, nucleic acid amplification, hybridization, mass spectrometry, nanostring, microfluidics, chemiluminescence, enzyme technologies and combinations thereof. Some of these technologies are described in later sections of the specification.

In one embodiment, a method of the disclosure for specifically detecting Cronobacter spp. may comprise identifying at least a first unique region specific to Cronobacter spp. referred to as a “first target nucleic acid sequence” for detection, obtaining or designing one or more primer pairs (polynucleotides) each primer pair comprising a “first primer” operable to hybridize to a first sequence within the first target nucleic acid sequence and at least a “second primer” operable to hybridize to a second sequence within the first target nucleic acid sequence; hybridizing at least a first pair to the first target nucleic acid sequence; amplifying the first target nucleic acid sequence to form a first amplified target nucleic acid sequence product; and detecting the at least first amplified target nucleic acid sequence product, wherein detection of the at least first amplified target nucleic acid sequence product is indicative of the presence of Cronobacter spp. In some embodiments, the method is also indicative of the absence of Enterobacter in the sample and/or the absence of non-Cronobacter spp. bacteria.

In some embodiments, a method as described above may further comprise: identifying at least a second target nucleic acid sequence specific to Cronobacter spp.; hybridizing a second pair of polynucleotide primers to the second target nucleic acid sequence; amplifying the second target nucleic acid sequence to form a second amplified target nucleic acid sequence product; and detecting the second amplified target nucleic acid sequence product, wherein detection of the second amplified target nucleic acid sequence product is indicative of the presence of Cronobacter spp. In some embodiments, the detection of the first and second amplified target nucleic acid sequence product indicates the presence of Cronobacter spp. Multiple targets nucleic acids may be amplified and identified to increase the specificity of the assay if desired. In some examples multiple target detection can be performed simultaneously on a sample (such as by a multiplex PCR method), or sequentially on a sample, or by splitting a sample into parts and processing parts in parallel with different sets of probes and primers.

In some embodiments, a first target nucleic acid sequence specific to Cronobacter spp. and a second target nucleic acid sequence specific to Cronobacter spp. may comprise one or more sequences such as but not limited to: SEQ ID NO: 1, SEQ ID NO: 2, SEQ ID NO: 3, SEQ ID NO: 4, SEQ ID NO:5, SEQ ID NO: 6, SEQ ID NO: 7, SEQ ID NO: 8, SEQ ID NO: 9, SEQ ID NO: 10, SEQ ID NO:11, SEQ ID NO: 12, fragments thereof, at least 25 nucleotide sequences thereof, complements thereof and sequences comprising at least 90% nucleic acid sequence identity thereof.

The first primer pair and the second primer pair of the methods, in some embodiments, may be one or more of: SEQ ID NO:13, SEQ ID NO:14, SEQ ID NO:15, fragments thereof, at least 10 contiguous nucleotide sequences thereof complements thereof, and labeled derivatives thereof.

In some embodiments, detection of an amplified target nucleic acid sequence product (such as a first amplified target nucleic acid sequence product and/or a second amplified target nucleic acid sequence product) as set forth in the embodiment methods described above may comprise use of a probe. Exemplary probes may comprise but are not limited to one or more sequences such as SEQ ID NO:13, SEQ ID NO:14, SEQ ID NO:15, fragments thereof, at least 10 contiguous nucleotide sequences thereof complements thereof, and labeled derivatives thereof.

Labeled probes and/or primers are helpful in detection and quantitation methods. Label for primers and probes may comprise at least one of the following: a dye, a radioactive isotope, a chemiluminescent label, a fluorescent label, a bioluminescent label, and an enzyme. Dye's may comprises a fluorescein dye, a rhodamine dye, and/or a cyanine dye. Some probes and primers may be dually labeled. Non-limiting examples of nucleic acid dyes include ethidium bromide, DAPI, Hoechst derivatives including without limitation Hoechst 33258 and Hoechst 33342, intercalators comprising a lanthanide chelate (for example but not limited to a nalthalene diimide derivative carrying two fluorescent tetradentate β-diketone-Eu³⁺ chelates (NDI-(BHHCT-Eu³⁺)₂), (See, e.g., Nojima et al., Nucl. Acids Res. Supplement No. 1, 105-06 (2001)), ethidium bromide, and certain unsymmetrical cyanine dyes such as SYBR® Green, PicoGreen®, and BOXTO dyes. SYBR Green dye is an “intercalating dye” which, as used herein, refers to a fluorescent molecule that is specific for a double-stranded polynucleotide or that at least shows a substantially greater fluorescent enhancement when associated with a double-stranded polynucleotide than with a single-stranded polynucleotide. Typically nucleic acid dye molecules associate with double-stranded segments of polynucleotides by intercalating between the base pairs of the double-stranded segment, by binding in the major or minor grooves of the double-stranded segment, or both.

Various embodiments of the present teachings relate to a multi-primer assay for detecting Cronobacter spp. in a sample. Methods of the disclosure, in some embodiments, comprise amplification methods that yield one or more amplification products. In some embodiments an amplification product may be detected by a real-time assay. A real-time assay may be, but is not limited to a SYBR® Green dye assay or a TaqMan® assay.

In embodiments of methods where more than one (e.g., two) amplification products may be formed, detection of a first amplification product may entail the use of a first probe and detection of a second amplification product may entail the use of a second probe. In such embodiments, a first probe may have a first label and a second probe may comprise a second label. In one example embodiment, a first probe may be labeled with a FAM™ dye and a second probe may be labeled with VIC® dye. In some embodiments, hybridizing and amplifying with a first pair of polynucleotide primers may be carried out in a first vessel and hybridizing and amplifying with a second pair of polynucleotide primers may be carried in a second vessel. In some embodiments, hybridizing and amplifying with a first pair of polynucleotide primers and hybridizing and amplifying with a second pair of polynucleotide primers may be carried out in a single vessel. In some embodiments, detection of amplified products may be by a real-time assay such as a SYBR® Green dye assay or a TaqMan® assay.

In some embodiments, the present disclosure describes methods based on utilizing whole-genome sequencing of a bacterium(s) and/or bacterial strain(s) of interest (e.g., unknown strains of Cronobacter spp.) and comparison to other known bacterial organisms (e.g., two known strains of Cronobacter spp.) to identifying the bacterium of interest.

For example, some embodiments of the disclosure describe assays to distinguish Cronobacter spp. from Enterobacter. Enterobacter is a known pathogen that is highly similar at the nucleotide level to the Cronobacter spp. serotype. Tests to detect Enterobacter often cross detect Cronobacter spp., thereby picking up false positives. The present disclosure provides nucleotide sequence information that may be used to design specific tests for the distinct detection of Enterobacter that does not cross-detect Cronobacter spp. For example, in some embodiments, using the genome sequence of Cronobacter spp. as described herein and the genomic sequence of Enterobacter, primers and probes may be designed that detect sequences unique to Enterobacter that are not present in Cronobacter spp.

In some embodiments, the present disclosure describes methods to selectively detect a particular species of Cronobacter spp. The present disclosure provides nucleotide sequence information that is unique to each of the six species of Cronobacter spp. The unique nucleotide fragments are listed SEQ ID NOs:16-1278. The detection of a particular species can be carried as described in the preceding discussion. Primers and probes may be designed using the unique species specific sequences to detect a particular Cronobacter species present in a given sample using the methods described above.

In other embodiments, a specific testing method may comprise: testing a sample that has been detected to be positive for Enterobacter comprising: a) providing an isolated nucleotide sequence of an Cronobacter spp.-specific nucleotide sequence such as but not limited to at least one nucleic acid sequence having the sequence of SEQ ID NO: 1, SEQ ID NO: 2, SEQ ID NO: 3, SEQ ID NO: 4, SEQ ID NO:5, SEQ ID NO: 6, SEQ ID NO: 7, SEQ ID NO: 8, SEQ ID NO: 9, SEQ ID NO: 10, SEQ ID NO:11, SEQ ID NO: 12, a fragment of the foregoing nucleic acids (also referred to as fragments thereof), a nucleic acid having at least 25 nucleotides of contiguous sequences of the foregoing sequences, complements thereof and/or sequences comprising at least 90% nucleic acid sequence identity thereof; b) contacting the at least one Cronobacter spp.-specific isolated nucleotide sequence with the sample; and c) detecting hybridization of the at least one Cronobacter spp. specific nucleotide sequence to a complementary nucleotide sequence in the sample. Detecting one or more nucleotide sequences that are unique to Cronobacter spp. are indicative that the test sample contains Cronobacter spp. Several exemplary detecting methods that may be used have been described in sections above. Embodiments of the disclosure also describe quantitative assays by which one of skill in the art, in light of this disclosure, may quantify the amount of Cronobacter spp. in the sample. This may be compared to the quantity of Enterobacter detected in the sample to determine whether the sample is devoid of Enterobacter or is contaminated with a combination of Enterobacter and Cronobacter spp.

In other embodiments, a specific testing method may comprise: testing a sample that has been detected to be positive for Cronobacter spp. to detect a particular species of Cronobacter comprising: a) providing an isolated nucleotide sequence of a particular species of Cronobacter spp.-specific nucleotide sequence such as but not limited to at least one nucleic acid sequence having the sequence described in SEQ ID NOs:16-1278, a fragment of the foregoing nucleic acids (also referred to as fragments thereof), a nucleic acid having at least 25 nucleotides of contiguous sequences of the foregoing sequences, complements thereof and/or sequences comprising at least 90% nucleic acid sequence identity thereof; b) contacting the at least one Cronobacter spp. species-specific isolated nucleotide sequence with the sample; and c) detecting hybridization of the at least one Cronobacter spp. species specific nucleotide sequence to a complementary nucleotide sequence in the sample. Detecting one or more nucleotide sequences that are unique to that particular species of Cronobacter spp. are indicative that the test sample contains the particular species of Cronobacter spp. For example detecting at least one or more nucleic acid having nucleic acids described in SEQ ID NOs:16-117 is indicative that the test sample is contaminated with C. sakazakii; detecting at least one or more nucleic acids having nucleic acid sequences described in SEQ ID NOs: 118-204 is indicative that the sample is contaminated with C. turicensis; detecting at least one or more nucleic acid having nucleic acids described in SEQ ID NOs:205-273 is indicative that the test sample is contaminated with C. malonaticus; detecting at least one or more nucleic acid having nucleic acids described in SEQ ID NOs:274-685 is indicative that the test sample is contaminated with C. muytjensii; detecting at least one or more nucleic acid having nucleic acids described in SEQ ID NO: 686-820 is indicative that the test sample is contaminated with C. genomosp1; detecting at least one or more nucleic acid having nucleic acids described in SEQ ID NO: 821-1213 is indicative that the test sample is contaminated with C. dublinensis; detecting at least one or more nucleic acid having nucleic acids described in SEQ ID NO: 1214-1278 is indicative that the test sample is contaminated with C. sakazakii ST4 strain. Several exemplary detecting methods that may be used have been described in sections above. Embodiments of the disclosure also describe quantitative assays by which one of skill in the art, in light of this disclosure, may quantify the amount of the particular species Cronobacter spp. in the sample. This may be compared to the quantity of Cronobacter spp. detected in the sample to determine whether the sample is devoid of other species of Cronobacter or is contaminated with a combination of different species of Cronobacter spp.

In some embodiments, methods for distinguishing a bacteria from an Cronobacter spp. are described and may comprise analyzing the genome of the bacteria for the presence of a sequence selected from the group consisting of SEQ ID NO: 1, SEQ ID NO: 2, SEQ ID NO: 3, SEQ ID NO: 4, SEQ ID NO:5, SEQ ID NO: 6, SEQ ID NO: 7, SEQ ID NO: 8, SEQ ID NO: 9, SEQ ID NO: 10, SEQ ID NO:11, SEQ ID NO: 12, fragments thereof, at least 25 nucleotide sequences thereof and sequences comprising at least 90% nucleic acid sequence identity thereof. Such methods may be used to distinguish the presence of Cronobacter spp. from a bacterium of several species. For example, methods of the disclosure may be used to distinguish the presence of Cronobacter spp. from other Enterobacter. Methods of the disclosure may also be used to distinguish the presence of Cronobacter spp. from other bacteria and Enterobacter.

Methods of the disclosure may further comprise preparing a test sample for amplification prior to hybridizing and/or amplification and may include steps such as but not limited to (1) bacterial enrichment, (2) separation of bacterial cells from other components of the sample, (3) lysis of bacterial cells, and (4) nucleic acid extraction.

In various embodiments, a variety of methods for amplifying nucleic acid sequences may be employed. Amplification may be mediated by polymerase chain reaction, having at least a first pair of polynucleotide primers and in some embodiments at least a second pair of polynucleotide primers. Amplification methods include, but are not limited to, polymerase chain reaction (PCR), RT-PCR, asynchronous PCR (A-PCR), and asymmetric PCR (AM-PCR), strand displacement amplification (SDA), multiple displacement amplification (MDA), nucleic acid strand-based amplification (NASBA), and/or rolling circle amplification (RCA), transcription-mediated amplification (TMA). (See, e.g., PCR Technology: Principles and Applications for DNA Amplification (ed. H. A. Erlich, Freeman Press, NY, N.Y., 1992); PCR Protocols: A Guide to Methods and Applications (eds. Innis, et al., Academic Press, San Diego, Calif., 1990); Mattila et al., Nucleic Acids Res. 19, 4967 (1991); Eckert et al., PCR Methods and Applications 1, 17 (1991); PCR (eds. McPherson et al., IRL Press, Oxford); and U.S. Pat. Nos. 4,683,202, 4,683,195, 4,800,159 4,965,188 and 5,333,675 each of which is incorporated herein by reference in their entirety).

Nucleic acid amplification techniques are traditionally classified according to the temperature requirements of the amplification process. Isothermal amplifications are conducted at a constant temperature, in contrast to amplifications that require cycling between high and low temperatures. Examples of isothermal amplification techniques are: Strand Displacement Amplification (SDA; Walker et al., 1992, Proc. Natl. Acad. Sci. USA 89:392 396; Walker et al., 1992, Nuc. Acids. Res. 20:1691 1696; and EP 0 497 272, all of which are incorporated herein by reference), self-sustained sequence replication (3SR; Guatelli et al., 1990, Proc. Natl. Acad. Sci. USA 87:1874 1878), the Qβ replicase system (Lizardi et al., 1988, BioTechnology 6:1197 1202), and the techniques disclosed in WO 90/10064 and WO 91/03573.

Examples of techniques that require temperature cycling are: polymerase chain reaction (PCR; Saiki et al., 1985, Science 230:1350 1354), ligase chain reaction (LCR; Wu et al., 1989, Genomics 4:560 569; Barringer et al., 1990, Gene 89:117 122; Barany, 1991, Proc. Natl. Acad. Sci. USA 88:189 193), transcription-based amplification (Kwoh et al., 1989, Proc. Natl. Acad. Sci. USA 86:1173 1177) and restriction amplification (U.S. Pat. No. 5,102,784), and self-sustained sequence replication (Guatelli et al., Proc. Nat. Acad. Sci. USA, 87, 1874 (1990)) and nucleic acid based sequence amplification (NABSA). (See, U.S. Pat. Nos. 5,409,818, 5,554517 and 6,063,603). The latter two amplification methods include isothermal reactions based on isothermal transcription, which produce both single-stranded RNA (ssRNA) and double-stranded DNA (dsDNA) as the amplification products in a ratio of about 30 or 100 to 1, respectively.

Other exemplary techniques include Nucleic Acid Sequence-Based Amplification (“NASBA”; see U.S. Pat. No. 5,130,238), and Rolling Circle Amplification (see Lizardi et al., Nat Genet. 19:225 232 (1998)). Amplification primers comprising nucleic acid sequences unique to Cronobacter spp. and/or designed based on these unique Cronobacter spp. sequences of the present disclosure may be used to carry out, for example, but not limited to, PCR, SDA or tSDA.

PCR is an extremely powerful technique for amplifying specific polynucleotide sequences, including genomic DNA, single-stranded cDNA, and mRNA among others. Various methods of conducting PCR amplification and primer design and construction for PCR amplification using sequences disclosed in this specification are described in the present disclosure. Generally, in PCR a double-stranded DNA to be amplified is denatured by heating the sample. New DNA synthesis is then primed by hybridizing primers to one or more target sequence(s) in the presence of DNA polymerase and excess dNTPs. In subsequent cycles, the primers hybridize to the newly synthesized DNA to produce discreet products comprising the primer sequences at either end. These amplified products accumulate exponentially with each successive round of amplification. The DNA polymerase used in PCR is often a thermostable polymerase. This allows the enzyme to continue functioning after repeated cycles of heating necessary to denature the double-stranded DNA for allowing primer annealing. Polymerases that are useful for PCR include, but are not limited to, Taq DNA polymerase, Tth DNA polymerase, Tfl DNA polymerase, Tma DNA polymerase, Tli DNA polymerase, and Pfu DNA polymerase. There are many commercially available modified forms of these enzymes including: AmpliTaq® and AmpliTaq Gold® both available from Applied Biosystems. Many are available with or without a 3′ to 5′ proofreading exonuclease activity. See, for example, Vent® and Vent®. (exo-) available from New England Biolabs.

Amplified products may be detected using probes or labeled primers. Since primers are incorporated into the ends of an amplicon, in some embodiments, labeled probes that are complementary to the primer sequences may be used. Alternatively labeled probes may be used for detection. Several other methods for the detection of an amplified product (e.g., PCR amplification product) include, but are not limited to, gel electrophoresis, capillary electrophoresis, and are known to one of skill in the art and may be applicable in light of the teachings of the present disclosure.

The disclosure also describes kits for the detection of Cronobacter spp. A kit of the disclosure may comprise at least one pair of amplification primers (e.g., PCR primers) that may be designed or derived from nucleic acid sequences of SEQ ID NO: 1, SEQ ID NO: 2, SEQ ID NO: 3, SEQ ID NO: 4, SEQ ID NO:5, SEQ ID NO: 6, SEQ ID NO: 7, SEQ ID NO: 8, SEQ ID NO: 9, SEQ ID NO: 10, SEQ ID NO:11, SEQ ID NO: 12, fragments thereof, complementary sequences thereof, sequences comprising at least 90% nucleic acid sequence identity thereof and complementary sequences comprising at least 90% nucleic acid sequence identity thereof. In some embodiments, the primers of a kit may be labeled. A kit comprising two (or more) pairs of primers may have primer pairs labeled with at least two (or more) different labels that may be detectable separately. A kit may further comprise at least one probe designed and/or derived from nucleic acid sequences comprising SEQ ID NO: 1, SEQ ID NO: 2, SEQ ID NO: 3, SEQ ID NO: 4, SEQ ID NO:5, SEQ ID NO: 6, SEQ ID NO: 7, SEQ ID NO: 8, SEQ ID NO: 9, SEQ ID NO: 10, SEQ ID NO:11, SEQ ID NO: 12, fragments thereof, complementary sequences thereof, sequences comprising at least 90% nucleic acid sequence identity thereof and complementary sequences comprising at least 90% nucleic acid sequence identity thereof. Probes comprised in kits of the disclosure may be labeled. If a kit comprises multiple probes each probe may be labeled with a different label to allow detection of different products that may be the target of each different probe.

The disclosure also describes kits for the detection of particular species of Cronobacter from samples. For each species, the primers and probes are derived from the unique signature sequences of that species. For example, primers and probes designed to signature nucleic acids described in SEQ ID NOs:16-117 can be used in kits to detect and/or identify C. sakazakii; primers and probes designed to signature nucleic acids described in SEQ ID NOs: 118-204 can be used in kits to detect and/or identify C. turicensis; primers and probes designed to signature nucleic acids described in SEQ ID NOs:205-273 can be used in kits to detect and/or identify C. malonaticus; primers and probes designed to signature nucleic acids described in SEQ ID NOs:274-685 can be used in kits to detect and/or identify C. muytjensii; primers and probes designed to signature nucleic acids described in SEQ ID NO: 686-820 can be used in kits to detect and/or identify C. genomosp1; primers and probes designed to signature nucleic acids described in SEQ ID NO: 821-1213 can be used in kits to detect and/or identify C. dublinensis; primers and probes designed to signature nucleic acids described in SEQ ID NO: 1214-1278 can be used in kits to detect and/or identify C. sakazakii ST4.

A kit of the disclosure may comprise at least one pair of amplification primers (e.g., PCR primers) that may be designed or derived from nucleic acid sequences listed in SEQ ID NOs: 1-12 and/or SEQ ID NOs:16-1278, fragments thereof, complementary sequences thereof, sequences comprising at least 90% nucleic acid sequence identity thereof and complementary sequences comprising at least 90% nucleic acid sequence identity thereof. In some embodiments, the primers of a kit may be labeled. A kit comprising two (or more) pairs of primers may have primer pairs labeled with at least two (or more) different labels that may be detectable separately. A kit may further comprise at least one probe designed and/or derived from nucleic acid sequences of SEQ ID NOs: 1-12 and/or SEQ ID NOs:16-1278, fragments thereof, complementary sequences thereof, sequences comprising at least 90% nucleic acid sequence identity thereof and complementary sequences comprising at least 90% nucleic acid sequence identity thereof. Probes comprised in kits of the disclosure may be labeled. If a kit comprises multiple probes each probe may be labeled with a different label to allow detection of different products that may be the target of each different probe.

In some embodiments, a kit for the detection of Cronobacter spp. may comprise: at least one pair of amplification primers (e.g., PCR primers) and/or at least one probe designed and/or derived from nucleic acid sequences comprising SEQ ID NO:13, SEQ ID NO:14, SEQ ID NO:15, fragments comprising at least 10 contiguous nucleotide sequences thereof and complements thereof. In some embodiments, kit primers may be labeled. A kit comprising multiple pairs of primers may have primer pairs each labeled with different labels that may be detectable separately. Probes comprised in kits of the disclosure may be labeled. If a kit comprises multiple probes each probe may be labeled with a different label to allow detection of different products that may be the target of each different probe.

A kit of the disclosure may further comprise one or more components such as but not limited to: at least one enzyme, dNTPs, at least one buffer, at least one salt, at least one control nucleic acid sample, loading solution for preparation of the amplified material for electrophoresis, genomic DNA as a template control, a size marker to insure that materials migrate as anticipated in a separation medium, and an instruction protocol and manual to educate a user and limit error in use. It is within the scope of these teachings to provide test kits for use in manual applications or test kits for use with automated sample preparation, reaction set-up, detectors or analyzers. In some embodiments, a kit amplification product may be further analyzed by methods such as but not limited to electrophoresis, hybridization, mass spectrometry, nanostring, microfluidics, chemiluminescence and/or enzyme technologies.

Components of kits may be individually and in various combinations comprised in one or a plurality of suitable container means.

In certain embodiments, the present disclosure describes a computer program product which includes a tangible computer-readable storage medium whose contents include a program with instructions being executed on a processor so as to perform a method for PCR data analysis. PCR data obtained for any PCR method such as but not limited to detecting the presence of a pathogen in a sample, detecting or diagnosing a pathogen and its cause, quantifying the amount of a pathogen contaminant in a sample may be analyzed by the present methods. A computer method for PCR data analysis of the disclosure can be performed by a system comprising one or more software modules. In some embodiments, the present disclosure describes methods for standardization of real-time analysis during a polymerase chain reaction (PCR). In some embodiments the present disclosure describes computer software algorithms and computer software based methods for standardizing analysis of data obtained during PCR and real-time PCR. Data as described in a method of the disclosure may be PCR data.

In some embodiments PCR data may be visualized in a two-dimensional plot of fluorescence intensity (y-axis) vs. cycle number α-axis). PCR data or a PCR data set may be transformed to produce a partition table of data points with one column including the fluorescence at cycle n, y(n), and a second column including the fluorescence at cycle (n+i), y(n+i), where i is typically 1 or greater. This 2-d plot is also referred to as an Amplification Plot.

There is an increasing need for better and more standardized analysis methods for analyzing real-time PCR data. Many inexperienced users are unsure how to set threshold values in order to obtain accurate Ct (cycle threshold) values. The term “Ct” represents the PCR “cycle” at which a signal first crosses above a fluorescence threshold which is set at a level above background noise. This “cycle” is not necessarily an integral number. Prior to this point the concentration of amplified product is considered too low to be of any significance. One of skill in the art will recognize that Ct is sometimes also known as Cq (quantification cycle) (See for example, Stephen Bustin et. al. “MIQE Guidelines: Minimum Information for Publication of Quantitative Real-Time PCR Experiments,” Clinical Chemistry 55:4; pages 611-622 (2009)). Inconsistent guidelines to set threshold values lead to large amount of variation between labs making PCR data comparison difficult. In some embodiments the present disclosure describes computer software algorithms and computer software based methods for standardizing analysis of data obtained during real-time PCR (PCR data).

In some embodiments, a software method and/or a computer based method and/or a computer implemented method of the disclosure is called a control-based threshold (CBT) method which is a method whereby a user uses a pre-defined percentage of a positive controls maximum plateau value (dRN) to set a threshold. Rn (normalized reporter) is the fluorescence emission intensity of a reporter dye divided by the fluorescence emission intensity of a passive reference dye. In some non-limiting example embodiments, ROX (6-Carboxyl-X-Rhodamine) may be used as a passive reference dye. The dRN (delta Rn) is the magnitude of the fluorescence signal generated during a PCR at each time point. A dRN value is determined by the formula:

dRN=Rn−(Baseline or Background)

In some embodiments, the disclosure describes a threshold setting method for analysis of PCR data. Threshold is described as the line whose intersection with the Amplification plot defines the Ct Improper threshold settings can lead to incorrect data interpretation and diminished reproducibility if set too low. A method of threshold setting may comprise setting an “optimal threshold setting” wherein an optional threshold setting should provide optimal sensitivity and specificity for a PCR assay.

Threshold setting methods generally comprise an Auto Ct method which typically uses a software algorithm to determine a threshold based on local signal characteristics or may comprise an Absolute Threshold method wherein a user chooses a single, absolute value to use for the threshold setting for all runs. A control-based threshold setting method, as described in the present disclosure, is based on a pre-defined percentage of a positive control plateau value. For example, a pre-defined percentage of a positive control's maximum plateau value (called dRN) may be determined at cycle 40 of a PCR reaction by a PCR instrument. This pre-defined percentage of dRN calculation may be entered by a user to set a threshold. A baseline may be set according to user instructions.

However, one of skill in the art in view of the present teachings will know that a positive control's maximum plateau value (dRN) may be determined at any cycle other than cycle 40 as well. For example, 40 cycles may be used by a user if the PCR reactions are run for a total of 40 PCR cycles. Accordingly, dRN may be calculated at the last cycle number that a PCR reaction is run for, such as but not limited to 40, 45, 50, or any other cycle number. Alternatively, dRN may be calculated as the average or median value of cycles near or at the end of the PCR reaction.

A control based threshold setting method of the present disclosure, according to some embodiments, may provide one or more advantages outlined below such as but not limited to:

increased consistency between real-time runs, between labs, and between different users (e.g. technicians)

the percentage of the dRN value can be customized for each product (such as a microbial diagnostic product, i.e., detecting Cronobacter and/or for a veterinary diagnostic product)

it takes into account the effects of a large proportion of chemical and/or instrument interactions that may influence Ct values

is compatible with all assays

Accordingly, a CBT method of the disclosure provides a balance of high analytical sensitivity and consistent target nucleic acid amplification across multiple Real Time PCR assays.

A control based threshold method to obtain Ct values may comprise: executing a set of computer readable instructions by a computer system interfacing with a PCR instrument comprising: 1) computing Rn values by dividing fluorescence values gathered by the PCR instrument for the dye associated with the target nucleic acid by that for the passive reference dye; 2) calculating a regression line to Rn values gathered by the PCR instrument during early PCR cycles (for example, cycles 3 to 10); 3) subtracting the regression line (the baseline) from Rn values to yield dRN values for all samples at all cycles; 4) obtaining an average of software derived values for dRN at the final PCR cycle for all positive control samples; 5) calculating a pre-defined percentage of the average dRN value; and 6) using the pre-defined percentage of the average dRN value as a threshold (this is the CBT) to determine Ct values by finding the intersection between the CBT and the dRN curve using a suitable interpolation method (such as linear or spline interpolation).

In some embodiments, a computer-implemented method of the disclosure for determining a control based threshold (CBT), may comprise: executing a set of computer readable instructions by a computer system interfacing with a PCR instrument comprising: 1) receiving (e.g., importing or inputting) polymerase chain reaction (PCR) data is into a computer program; computing a dRn value using the PCR data; and computing a Ct (cycle threshold) value using an interpolation algorithm to determine the intersection between the dRn and the cycle number of the PCR reaction from where PCR data is obtained; using the computed Ct value as the control based threshold value for all PCR reactions.

A method for setting a control based threshold CBT may in some embodiments comprise: executing a set of computer readable instructions by a computer system interfacing with a PCR instrument comprising: 1) exporting a positive control's maximum plateau value (a dRN value) for positive control samples of a PCR reaction wherein the dRN value is an average of software derived values for each respective dRN at the final PCR cycle for all positive control replicates; 2) calculating a pre-defined percentage of the dRN value; and 3) using the pre-defined percentage of the average dRN value as a threshold (CBT), to determine a Ct (cycle threshold) value for all samples. In some embodiments a method may further comprise: receiving polymerase chain reaction (PCR) data is into a computer program; computing a dRn value using the PCR data; and computing a Ct value using an interpolation algorithm to determine the intersection between the CBT and the dRN data; and optionally comparing to Ct values of a negative control, a positive control, and/or an internal positive controls.

In some embodiments of the each dRN value is the magnitude of fluorescence signal generated during PCR by a positive control at the last PCR cycle number and is determined by the formula: dRN, Rn−(Baseline or Background), wherein, Rn is the fluorescence emission intensity of a reporter dye divided by the fluorescence emission intensity of a passive reference dye and Baseline or Background is a linear regression line fit to Rn data within a pre-determined range of PCR cycles.

In some embodiments, wherein a dRN value of a positive control used is the average or median across positive controls of the average or median dRN values over a pre-determined range of PCR cycles near the end of the PCR data.

In some embodiments wherein the Ct value derived from the CBT and dRN data is compared to two or more Ct value ranges where each range is associated with a biologically meaningful diagnosis such as positive, suspected positive, and negative outcomes.

In some embodiments wherein the Ct value derived from the CBT and dRN data of positive and negative controls is used to determine quality control status such as the presence of PCR inhibition.

In summary a CBT method of the disclosure provides: 1) complete, clear and concise instructions provided to a Real Time PCR user including instructions for one or more of the following non-limiting examples such as, a) baseline settings, b) threshold settings and/or c) end-user controls; 2) a consistent threshold methodology for a) between real time PCR runs, different laboratories and different users (different level of user knowledge); and is generally b) compatible with all assays and has c) no change in positive or negative calls between various users.

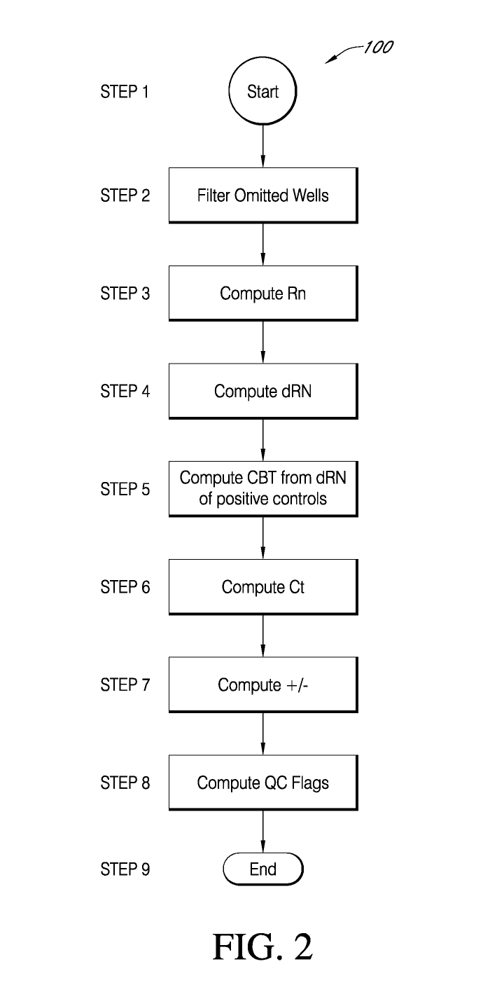

FIG. 2 is a schematic diagram of an algorithm 100 comprising one or more software modules that perform a method for PCR data analysis, in accordance with certain embodiments. As shown in FIG. 2, a CBT method algorithm 100 may start at step 1 comprising a start step, wherein data is entered or imported into a computer program through a PCR machine. In Step 2 of algorithm 100, data may be filtered, to filter out omitted wells, such as sample wells that do not have any samples or wells otherwise omitted from further analysis by the user. Step 3 may comprise computing the dRn value. Step 4 may comprise computing the baseline and using it to get dRN values. Step 5 may comprise computing the CBT from dRN values of Positive Control samples. Step 6 may comprise computing the Ct value by applying any suitable interpolation algorithm (e.g., linear or spline interpolation) to find the intersection between the CBT and the dRN values. Step 7 may comprise using the Ct values to call out positive (+) and negative (−) values associated with detection or non-detection of a PCR amplified nucleic acid which is described as “compute+/−.” Ct values that are small are “positives”; large values are “negatives” and in-between values are considered to be “suspected positives.”

In Step 8 Quality Control check may be performed by examining the Ct values of controls (negative controls, positive controls, internal positive controls, etc.) and signal characteristics (for e.g., low ROX values).

The program ends in Step 9 and data is outputted for an end user to view regarding detection or non-detection as well as in some embodiments quantitation of detected amplified product.

The present disclosure in some embodiments describes a collection of software modules that facilitate presence-absence based identification of data obtained by PCR methods. For example, a software method of the disclosure may be operable to analyze PCR data to enable an end user to know whether a microorganism is present or absent in a sample. In another example, an end user may know if a certain nucleic acid is present or absent in a sample.

In some embodiments a software module of the disclosure may also facilitate quantification of microbes or certain nucleic acids that may be present in a sample, thereby providing quantitative analysis modules. In some embodiments, quantification may be done in real-time.

In some embodiments, software modules of the disclosure facilitate quantification using TaqMan® probes and qPCR for diagnostic purposes. Accordingly, in some embodiments, computer program product (software and/or algorithm) of the present disclosure includes a tangible computer-readable storage medium whose contents include a program with instructions being executed on a processor so as to perform a method for PCR data analysis thereby enabling a user, who may not have detailed PCR or data analysis knowledge, to reach a conclusion regarding the PCR-based biological test being performed. In some non-limiting example embodiments, a PCR-based biological test may be carried out using a Cronobacter testing kit of the disclosure. One of skill in the art will recognize that the computer methods and software is not limited to testing Cronobacter and may be used with any other PCR based testing kit and/or PCR testing composition for testing for the presence and or absence of any microbial, fungal, viral, animal, plant, insect or human nucleic acid and thereby may be used for a variety of applications including but not limited to, food safety testing, medical diagnostic, environmental testing, animal diagnostic testing and other applications.

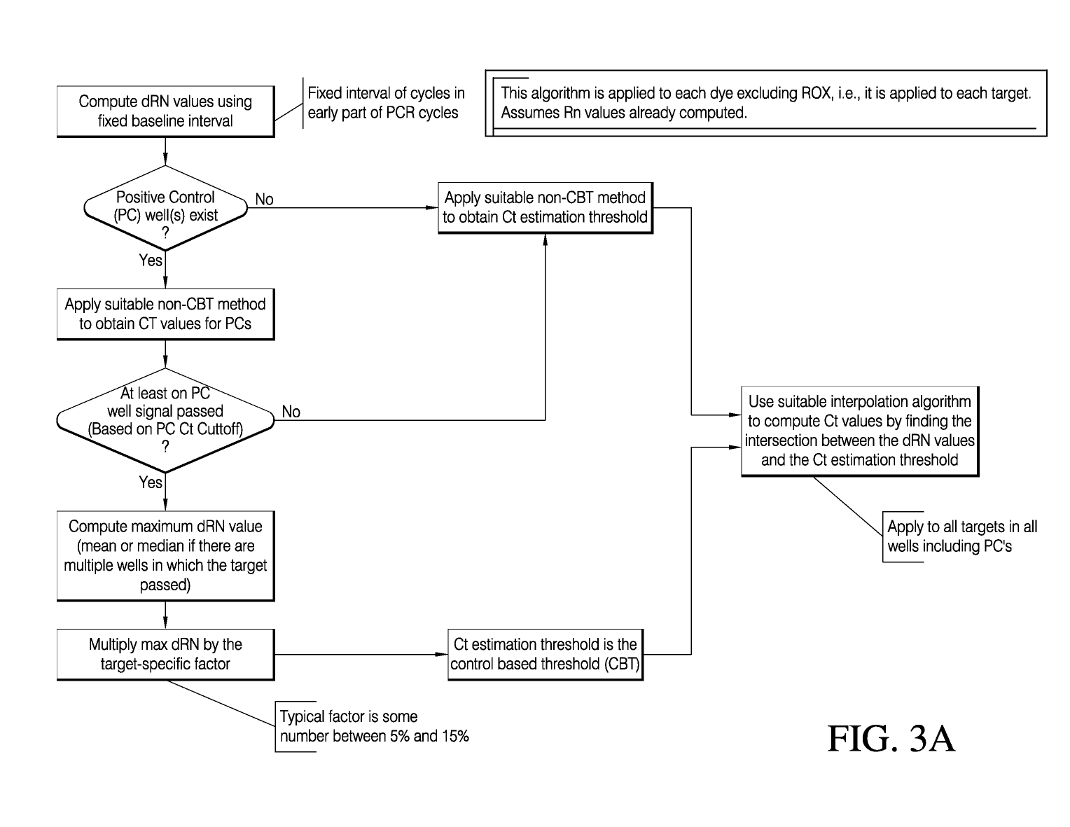

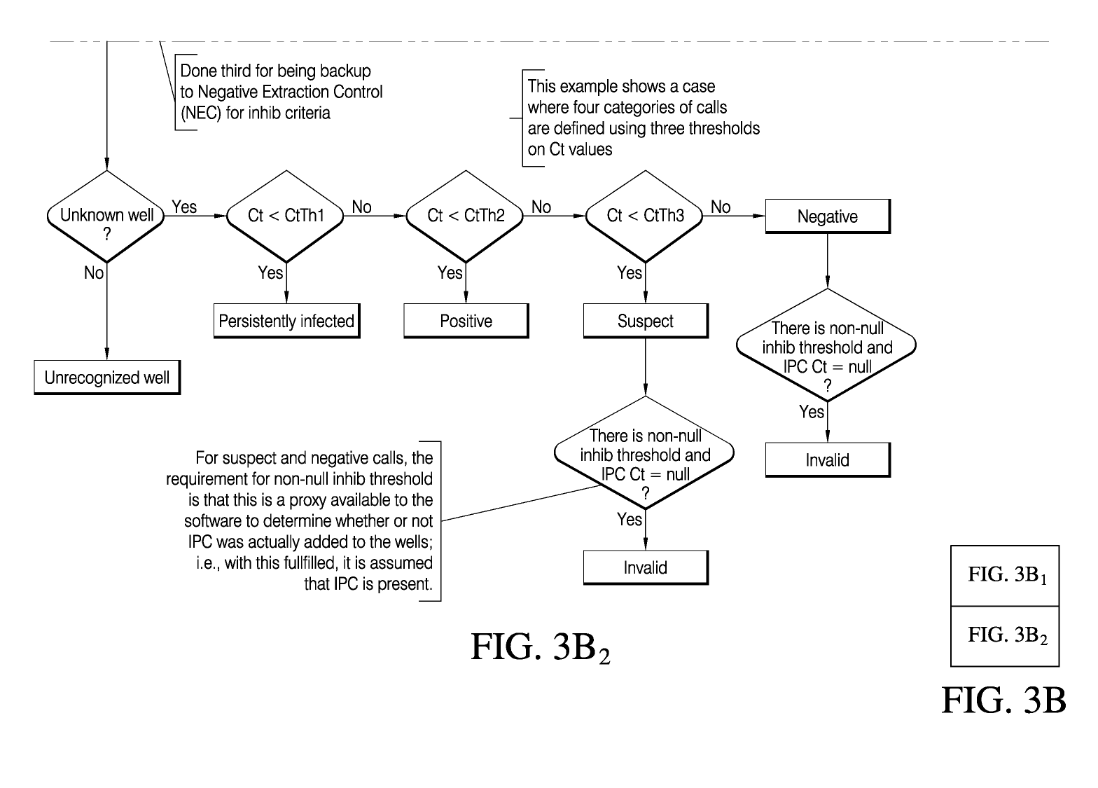

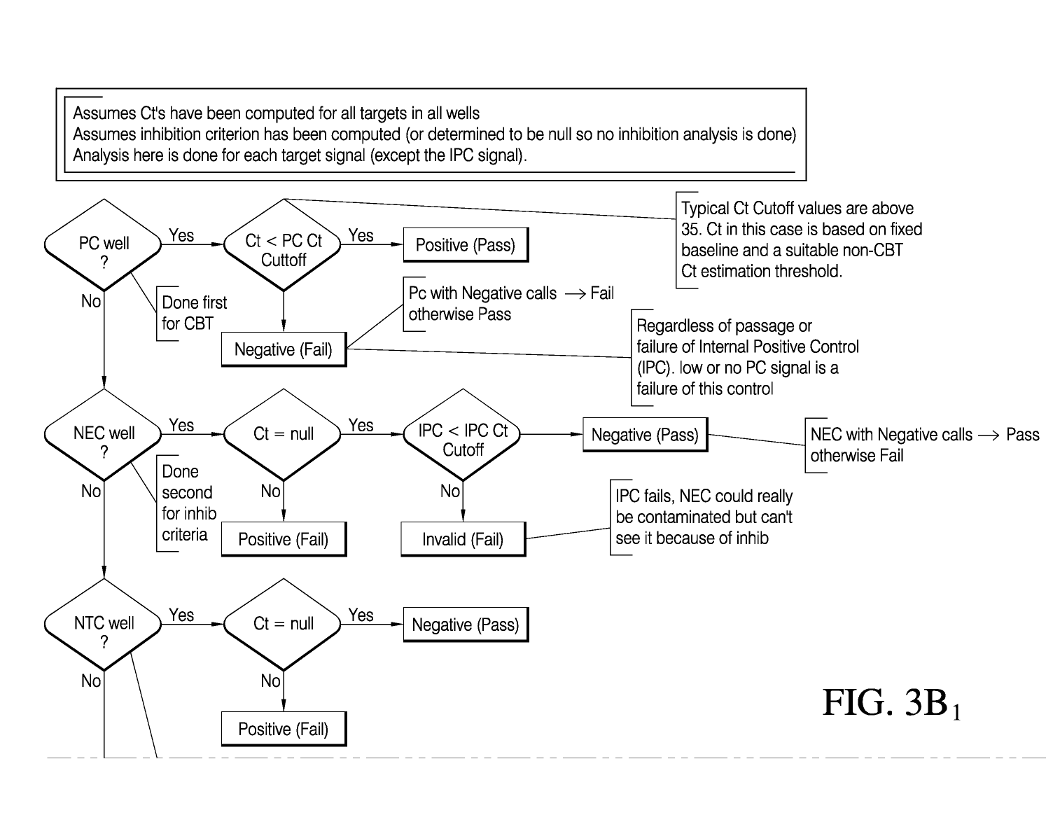

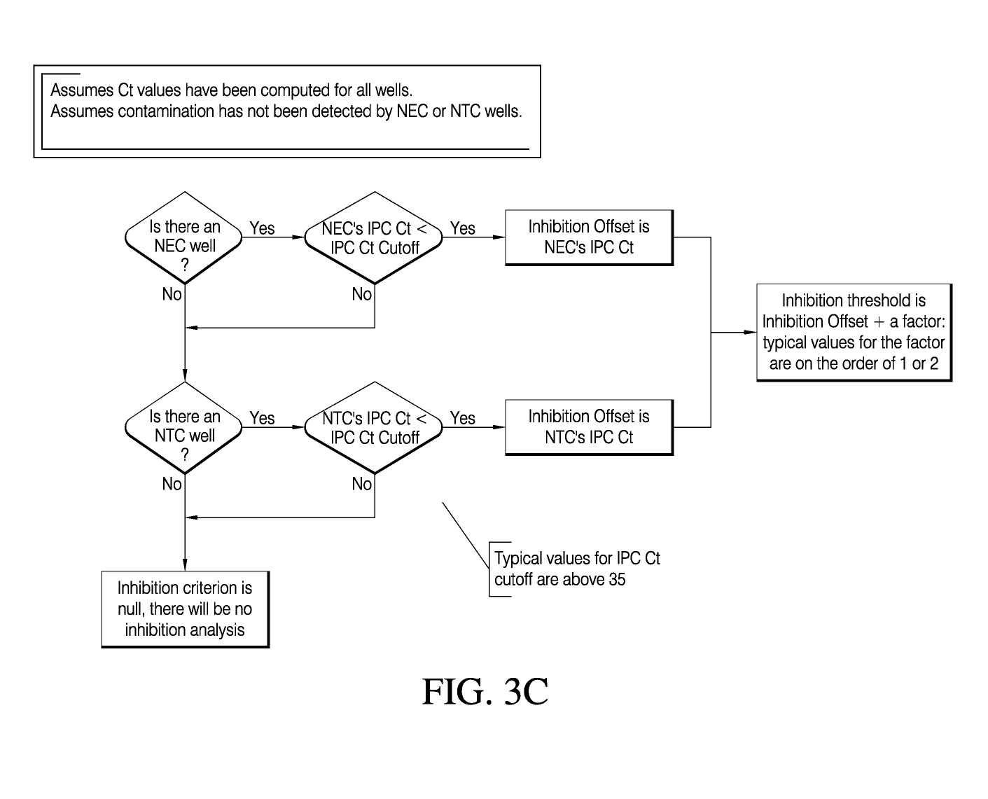

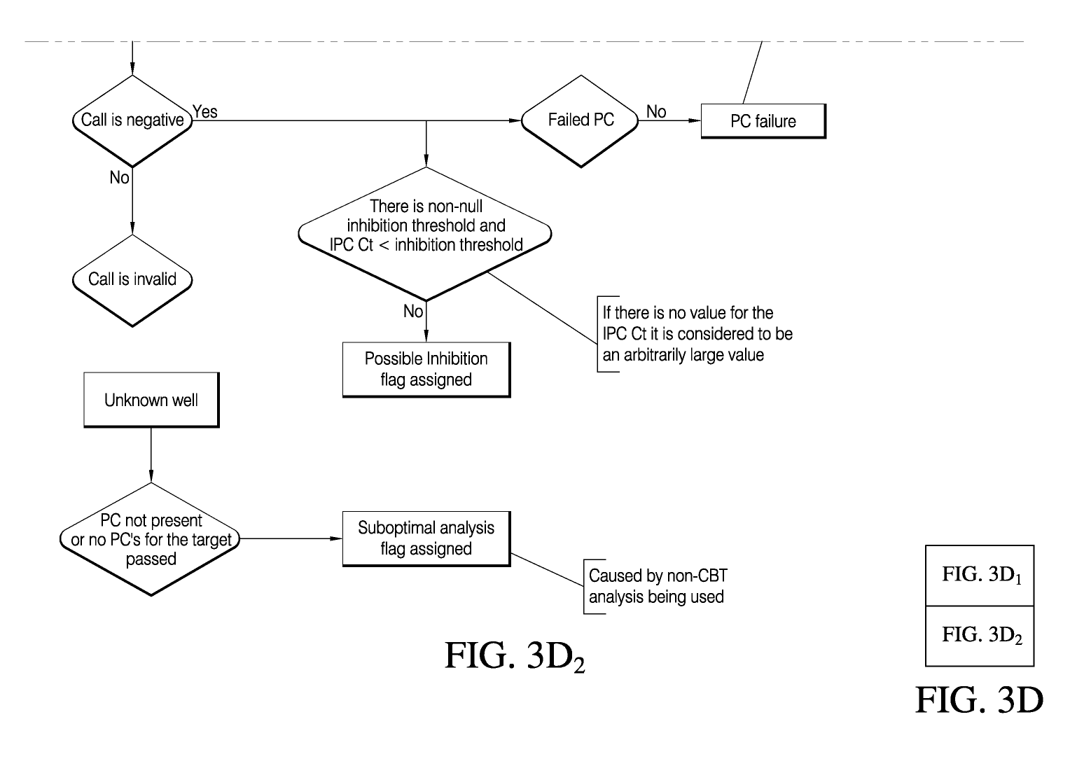

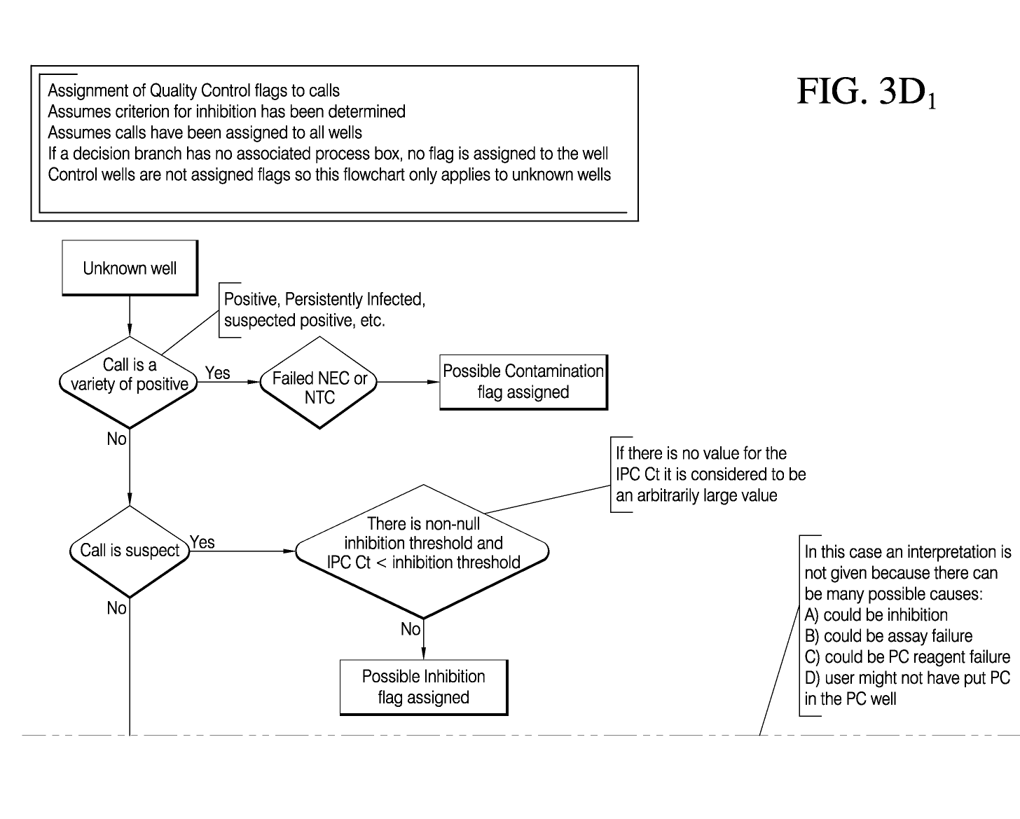

Algorithm 100 as shown in FIG. 2 may, in some embodiments, comprise a collection of software modules that facilitate analysis of data obtained by PCR methods. FIGS. 3A-D shows detailed schematic diagrams of various example algorithms and distinct software modules that may be comprised in algorithm 100.

Some definitions of terms used in depiction of FIGS. 3A to 3D are listed below:
PC: Positive Control, a sample that is known to contain the targeted genetic material
IPC: Internal Positive Control, a strand of DNA that is introduced into each sample's well. The reagents associated with the assays contain probes that target this strand of DNA.
NTC: No Template Control: a type of negative control. It is known to be devoid of any genetic material and is introduced to the assay workflow at the point of performing PCR.
NEC: Negative Extraction Control: a type of negative control. It is known to be devoid of the targeted genetic material and it is introduced to the assay workflow at the point of sample preparation.
Inhibition: The PCR reaction could not proceed as expected.
Null: The case where no Ct value is assigned. This may happen if, for example, the dRN data never intersects the Ct estimation threshold.
Ct estimation threshold: dRN level which is considered significantly above noise;
   the “cycle” at which dRN data first reaches this level defines the Ct value
Persistently Infected An example call category associated with very low Ct values.

An algorithm described in FIG. 3A is an algorithm for computing Ct; an algorithm for assessing if a sample is a positive or negative (+/−) is shown in FIG. 3B; an algorithm for determining inhibition criteria is shown in FIG. 3C and FIG. 3D shows an algorithm for Assigning QC flags. One of skill in the art, in light of this disclosure will recognize that the example algorithms shown in FIGS. 3A-3D are not limiting and additional/alternative algorithms may be comprised in algorithm 100. Algorithms shown in FIGS. 3A-3D shows methods of analysis in various workflows for detection of various types of PCR amplified products.

Software and algorithms of the disclosure may have one or more features described here. In some embodiments, a workflow may comprise software that adapts to a user is described. In some embodiments, a workflow of the disclosure adapts to a single-plate or multi-plate workflow based on how many data files a user imports into a software.

In some embodiments, a software method of the disclosure comprises defining assays within independent files that can be installed/uninstalled into/from the software application. This allows a validated software to continue support installed assays and support newly installed assays without needing to revalidate their entire computer system and assay installations that had already been validated.

In some embodiments, a software of the disclosure comprises modules for comparing amplification and/or multicomponent plots between an unknown sample and positive and negative controls and other samples when doing Quality Control to confirm or override calls made by the software.

In some embodiments, a software module of the present disclosure allows selection of a region of a plate by a simple key stroke or double clicking a pointing device associated with a computer. For plates like Open Array and TLDA cards, there are natural regions of wells. Users would be enabled to quickly select these regions of wells by double clicking on any of the constituent wells (single click->select well. Double click->select region of wells). Alternatively, the software can provide a zoom function to move between different resolutions of display. In each resolution a different sub-region of wells is represented by a single cell in the display. Any operations performed on a cell at a given resolution applies to all wells in the sub-region represented by that cell.

In some embodiments, a software module of the present disclosure allows direct editing of a cell with multiple attributes, by providing a selector to direct editing to one of the attributes when first entering a cell and, when within a cell, a simple method to navigate to other attributes (such as a carriage return).

In some embodiments, a software module of the present disclosure allows adding/removing custom attributes of a well within the software with the ability to manipulate them and assign values to them in the same manner as pre-existing attributes.

In some embodiments, a software module of the present disclosure allows a combined display of dRN and the un-altered data underlying dRN values (fluorescence values for each dye).

In some embodiments, a software module of the present disclosure collects together problem cases for quick navigation to the data underlying these samples. This facilitates the process of reviewing and annotating diagnostics results.

In some embodiments, a software module of the present disclosure allows for combining plates of data to analyze together as a unit, an analysis unit.

In some embodiments, a software module of the present disclosure allows applying controls from one plate over all the plates in the analysis unit.

In some embodiments, a software module of the present disclosure allows multiple results for a sample to be pooled together and fed to an algorithm such as an artificial neural network or other pattern recognition algorithm, to produce a final diagnostic result (can be multi-functional: e.g., copy number variation results, single nucleotide polymorphisms, protein quantification results, mRNA quantification results (gene expression), etc.

In some embodiments, a software module of the present disclosure allows presenting diagnostic results on a plate grid (spreadsheet).

In some embodiments, a software module of the present disclosure allows gray region for diagnostics (suspect positive region or suspect negative region, as well as something between positive and negative diagnoses).

In some embodiments, a software module of the present disclosure allows analyzing well contents to identify possible inhibition of PCR by an interfering substance (using an positive control internal to the well).

In some embodiments, a software module of the present disclosure allows analyzing well contents to identify possible inhibition of PCR by competition for reagents (there is one or more strongly dominant target(s) in the well which causes other targets to appear negative because they failed to compete for the PCR resources).

In some embodiments, a software module of the present disclosure allows subdividing the Ct range into categories and associating these with an approximate but meaningful quantification unit relevant to a diagnosis; e.g., high is from Ct=1 to 10, medium is Ct from 11 to 28, low is Ct from 29 to infinity. The software module allows calibrating to these levels by using samples for which the diagnostic level is known.

While the principles of inventions disclosed herein have been described in connection with specific embodiments, it should be understood clearly that these descriptions are made only by way of example and are not intended to limit the scope of inventions described herein. The present disclosure is for the purposes of illustration and description. It is not intended to be exhaustive or to limit disclosed embodiments to the precise forms as described. In light of this disclosure, many modifications and variations will be apparent to a practitioner skilled in the art. What is disclosed was chosen and described in order to best explain the principles and practical application of the disclosed embodiments of the art described, thereby enabling others skilled in the art to understand various embodiments and various modifications that are suited to contemplated uses. It is intended that the scope of what is disclosed be defined by the following claims and their equivalence.

### EXAMPLES

Some embodiments of the present disclosure may be understood in connection with the following examples. However, one skilled in the art will readily appreciate the specific materials, compositions, and results described are merely illustrative of the disclosure, and are not intended to, nor should be construed to, limit the scope of the disclosure and its various embodiments.

### Example I. Strain Selection

Nine strains representing each of the six named species of Cronobacter spp. were selected. The selected strains include three C. sakazakii (strain 680, 696, 701), two C. malonaticus (681, 507), one C. muytjensii (530), one C. turicensis (564), one C. dublinensis (582), and one C. genomosp1 (581) strain. Among these, strain 701 is a C. sakazakii ST4 strain that has been strongly associated with neonatal meningitis (Joseph and Forsythe, 2011). The C. sakazakii BAA-894 strain sequence was used as a reference for the 696, 701, 680, 507 and 681 strain sequences, and the C. turicensis z3032 strain sequence was used as a reference for the 564, 582, 530 and 581 strain sequences, due to their availability as finished genome sequences in the public databases. Table 1 depicts the information on nine newly assembled Cronobacter genomes and two publicly available Cronobacter genomes.

TABLE 1

| | | | Genome | MLST | Refseq |
|---|---|---|---|---|---|
| | | | size | Sequence | Accession |
| Species | Strain | Source | (Mbp)^{b} | Type | Num |
| C. sakazakii | 696 | Clinical | 4.90 | 12 | |
| C. sakazakii | 701 | Clinical | 4.75 | 4^{c} | |
| C. sakazakii | 680 | Clinical | 4.35 | 8 | |
| C. sakazakii | BAA- | Powered | 4.53 | 1 | NC_009778- |
| | 894^{a} | formula | | | NC_009780 |
| C. | 507 | Clinical | 4.45 | 11 | |
| malonaticus | | | | | |
| C. | 681 | Clinical | 4.50 | 7 | |
| malonaticus | | | | | |
| C. turicensis | 564 | Clinical | 4.50 | 5 | |
| | | (blood) | | | |
| C. turicensis | Z3032^{a} | Clinical | 4.60 | 19 | NC_013282- |
| | | | | | NC_013285 |
| C. | 582 | Unknown | 4.68 | 36 | |
| dublinensis | | | | | |
| C. muytjensii | 530 | Infant | 4.53 | 49 | |
| | | formula | | | |
| C. | 581 | Environ- | 4.45 | 30 | |
| genomosp. 1 | | mental | | | |

^{a}Strains for which the complete genome sequence is publicly available.
^{b}Sizes of newly sequenced genomes were derived from the sum of the length of all contigs from the de novo assembly.
^{c}C. sakazakii ST4 is strongly associated with neonatal meningitis (Joseph and Forsythe, 2011).

Table 2 depicts de novo assembly statistics for the nine Cronobacter strains sequenced.

TABLE 2

| | | | N50 of | | | | Estimated |
|---|---|---|---|---|---|---|---|
| | | Total cont | scaffolds | Num of | N50 of | Num of | num of |
| Species | Strain | length (bp) | (bp) | scaffolds | contigs (bp) | contigs | ORFs (bp) |
| C. sakazakii | 696 | 4,897,138 | 297,746 | 920 | 4,336 | 2,659 | 4,190 |
| C. sakazakii | 701 | 4,752,729 | 346,235 | 1,171 | 3,538 | 3,148 | 3,955 |
| C. sakazakii | 680 | 4,350,201 | 75,779 | 2,308 | 2,007 | 4,994 | NA |
| C. malonaticus | 507 | 4,447,701 | 373,979 | 464 | 3,703 | 2,361 | 3,727 |
| C. malonaticus | 681 | 4,496,745 | 345,762 | 263 | 5,537 | 1,592 | 3,884 |
| C. turicensis | 564 | 4,500,608 | 411,105 | 263 | 4,796 | 1,807 | 3,820 |
| C. dublinensis | 582 | 4,677,592 | 229,230 | 539 | 3,822 | 2,657 | 3,964 |
| C. muytjensii | 530 | 4,533,101 | 596,924 | 444 | 4,925 | 1,937 | 3,877 |
| C. genomosp. 1 | 581 | 4,450,737 | 331,248 | 389 | 4,506 | 2,085 | 3,867 |

### Example II. SOLiD™ Sequencing

Long mate-paired genomic DNA libraries with approximately 1.8 kb inserts were constructed for each strain from the isolated strain genomic DNA, and 2×50 bp reads were obtained from each pair. Sequencing was carried out to 2×50 base pairs using SOLiD™ chemistry (Applied Biosystems) according to the manufacturer's instructions.

Over 23-36 million reads, of approximately 500-800 fold coverage of the genomes, were obtained for each strain. The colorspace reads were error-corrected and then assembled using the SOLiD bacterial de novo assembly pipeline, which employs the velvet assembly engine (Zerbino and Birney, 2008). The nine genomes (696, 701, 680, 681, 507, 530, 564, 581, 582) (Table 2) were successfully de novo assembled into contigs and scaffolds. The ultimate genome assemblies contain 1600-5000 contigs with N50 of 2.0-5.5 kb and 260-2300 scaffolds with N50 of 76-600 kb.

### Example III. Sequencing and Assembly of Nine Cronobacter Strain Genomes

In some embodiments, the genomic sequence of the nine Cronobacter genomes have been sequenced and specific and unique regions identified.

Genomic DNA was isolated using Qiagen DNeasy Blood & Tissue Kit following the instruction from the manufacturer (Qiagen, Valencia Calif.). The isolated genomic DNA was used to construct long mate-pair libraries, which were sequenced to 2×50 base pairs using SOLiD™ chemistry (Applied Biosystems), according to the manufacturer's instructions (Example II).

Over 23-36 million reads, of approximately 500-800 fold coverage of the genomes, were obtained for each strain. The colorspace reads were error-corrected and then assembled using the SOLiD bacterial de novo assembly pipeline, which employs the velvet assembly engine. The nine genomes were successfully de novo assembled into contigs and scaffolds. The ultimate genome assemblies contain 1600-5000 contigs with N50 of 2.0-5.5 kb and 260-2300 scaffolds with N50 of 76-600 kb (Example III).

The assembled genome scaffolds were aligned to the most closely related public complete genomes using MUMmer (Kurtz et al., 2004). The scaffolds of strains 696, 701, 680, 507 and 681 were aligned to the C. sakazakii BAA-894 complete genome, and the scaffolds of 564, 582, 530 and 581 were aligned to the C. turicensis z3032 complete genome. Scaffolds were broken at points where non-contiguous regions of the reference genome were juxtaposed, and then re-ordered so that they were syntenic with the reference genome. All scaffolds from a given strain were concatenated into a single pseudogenome, which was then annotated at the RAST automated annotation server (Aziz et al., 2008).

By aligning the contigs against the public complete Cronobacter genomes followed by annotation using RAST, draft genomes were generated of size 4.4-4.9 Mb containing 3,700-4,200 annotated genes (Table 2). Genome comparison revealed an overall high sequence identity (89-98%) between the Cronobacter species but also suggested various degrees of divergence.

In some embodiments sequences specific and unique to and shared by the eleven Cronobacter spp. strains can be used to identify Cronobacter spp. or distinguish Cronobacter spp. from all other Enterobacter genomes. One example method used to identify Cronobacter spp. specific sequences is outlined in Example IV.

Embodiments of the present disclosure have identified serotype specific and unique DNA sequences of Cronobacter spp. shared by the eleven strains (e.g., but not limited to, SEQ ID NO:1, SEQ ID NO:2, SEQ ID NO:3, SEQ ID NO:4 SEQ ID NO:5, SEQ ID NO:6, SEQ ID NO:7, SEQ ID NO:8, SEQ ID NO:9, SEQ ID NO: 10, SEQ ID NO: 11 and SEQ ID NO:12) which were utilized for an assay design (described in Example VI) and the subsequent detection of Cronobacter spp. by amplification (PCR), hybridization and other molecular biology techniques as known to one skilled in the art.

Twelve Cronobacter spp. specific sequences, covering a sum of 2,070 nucleotides were found using the analysis of Example IV. These sequences are shown in SEQ ID NO: 1-12.

In some embodiments, the sequences designated by SEQ ID NO:1, SEQ ID NO:2, SEQ ID NO:3, SEQ ID NO:4 SEQ ID NO:5, SEQ ID NO:6, SEQ ID NO:7, SEQ ID NO:8, SEQ ID NO:9, SEQ ID NO: 10, SEQ ID NO: 11 and SEQ ID NO:12, are signature sequences against which Cronobacter spp.-specific diagnostic assays have been designed in the present disclosure. No comparable sequences were found in the GenBank database (release 183.0). The coordinates of Cronobacter spp. specific sequences are provided in Table 3.

TABLE 3
Exemplary Sequences and Coordinates Described in Some
Embodiments with Corresponding SEQ ID NOS:

>fragment_523060-523208
TGGGAATGTGAGCTCTGGGGCAGTCGTGGCTCCTTCCACAGCACCTGTTGTTAGCACGCA
AGAACCGACCGTAAGCAGCACGACCAATAGCGCACCTGTCGCTACCTGGCGCTGGCCGA
CTGAAGGCAACGTTATCGAGAACTTCTCCG
(SEQ ID NO: 1)
>fragment_687438-687541
AATGAAGCCCATCGCCCGACCCGCGCTCAGGACGCTATCGACGTTAAACTGCGCGCTTAA
AAAAATAATACGCTTTTCCCGAATCAGGCCAGGAAGCTCCGGCA
(SEQ ID NO: 2)
>fragment_2113821-2113966
TCGTCCTGGGAGTTTTGATTGGCCATAAGGTGACATCTCGGGGAGGTTTAAGCAGTTACA
TTACCTGCATTATTCTAACAAAACATTAACAGTAACGCGTACACTTTTGGTCTAAACTTAG
CACTGAAAATGCAGCTGACAAGCAA
(SEQ ID NO: 3)
>fragment_2740245-2740349
TTCTCCATACGTTTGCATTTTCACTACGTGATTGAAGCGAAAACCATACCATTAAAGGCG
CTATGCCGACAACAAACGCGGCGGAGGGTAAATGGTTTCACCGGG
(SEQ ID NO: 4)
>fragment_2823697-2824144
GGCGTTGCTGTTTCCATGTGTATCAATCCTTTACCCTGAATAATTGTTGTTTTTTTGTTGCT
TTTATCGCCAGAAAAAAAGCACAACGAAGAAAGTGTTCCAATTTACTGGTCAATAAAATA
GCATCTGATTTGCTTAGATATATTTATCGAAATTTGGTAGCAGAGACTTTAAATAATTTCG
TTTATCATTCTGTGCGACATTATTTTTAACGATTCAGCACCGGGAATAATTATTTCCGCGC
CGTTATTGTTTCTCATTTGAAACCGCCTTGTTAATTTATTGTATGTTATTTTCTCCCCGATA
TACTCAATTCCCGTCTTGACCTTACTTTACATAGGATTTTGTTATAACCCTTGCCATGTGGC
TGTCATGGCTTACATTTTACATTTTGTTGCATTGGCTGTGACGGTAGCGACAGAACCCGGT
TACACCCCGCAGACAGTGC
(SEQ ID NO: 5)
>fragment_3031790-3031935
CCACGCGGCATGGGCCGTGGTTTTTGCTCGCTTTGGTCTGCACAGCATAAAAGAAAGTGG
TATTCTCGGGCTATTGCCCAGGCCCGTCTTGCGGCACATCACAACGATAACCCAGAGGCC
CGCACGCCGCGCTGCACCGGGCCCAG
(SEQ ID NO: 6)
>fragment_3043432-3043664
CTTATTTGCTGTGCGCATGAGTACACATTAAGCAATCTTAAGTTTTTAGTGGCTATTTTG
CCGGACGATCCCGCTTTAACCCAATATTATCGAGAAGTTAATGAGTTACGTGCAAAAAAT
CAAAAAACACTACCCTCAATTCTTAAAAATGAGCGCCAAATAAATCTTTACCTGCGATTA
GAAGACGATGATTTAATTGATAAAATTAATCCAGATCTTCGGTTGTCCACTCC
(SEQ ID NO: 7)
>fragment_3084403-3084556
GCATAAAAAAGCCGTGACAACCACGGCTCGCCCGACAGGCCAGGCTCACCCACCCGTATT
CAGGTGGCGCAGACTATATCACCGAAGCAGAGCGCTCACAAATAACCTCGCCTTCACCGT
ATGATTAATCATCAGCTTGCGAGACGCGTCACCC
(SEQ ID NO: 8)
>fragment_3115773-3115905
TTAAGCGAGGCGCAGCGCAAGGGGGTGGAAGTGCTGGCCTGGAAGGCCTCGCTCTCCGC
CAGTGAGATAACGCTGACGTCGCCTTTGCCGGTTCGCTTATAACCAGTTGATCCGCAATC
GACTAATAATGATA
(SEQ ID NO: 9)
>fragment_3284085-3284250
GCCAGCCACCGCCAGACGACCTGTCTGGATGTTCCCACAATCCTTCCTCATGTTAATTGA
CTGACCGGCAGCCTCATGCCGCCTGAAATTCTCAGATACTTCATGCTAACCAGGCGAAGG
CCGTTGCGCCATGTCGCGAACATTTTTTTACCATTCGCGCATTAAT
(SEQ ID NO: 10)
>fragment_3640902-3641007
GCGCGCATGCAACAAAAAAATTGCTTAATCCGCTCTCTTGCTCACATTTTGCGCATCAAC
GCGCATTTCTGATGCCTTTTCAGCCACTCATGGTGAAATAATCCAC
(SEQ ID NO: 11)
>fragment_4359424-4359603
GTCGATTCGTTGTGCATGATGTTTCCCCCTTTGCGTCGCGATTCTACGCAACTTTTCCGG
ATTCTGCCGGGTTGCTTCAACAGAAAGAAACTTATTTAAACAAATTAAGTCTGAAATAAC
GCCCGAAACGGAAAAGTGGTTATGTTGATTTCCGCTGCGACGTTTTATAGTACGACTTTC
(SEQ ID NO: 12)

Any of the sequences SEQ ID NO:1, SEQ ID NO:2, SEQ ID NO:3, SEQ ID NO:4 SEQ ID NO:5, SEQ ID NO:6, SEQ ID NO:7, SEQ ID NO:8, SEQ ID NO:9, SEQ ID NO: 10, SEQ ID NO: 11, SEQ ID NO:12, as well as complements and sequences comprising at least 90% nucleic acid sequence identity thereof can be used to identify and/or distinguish Cronobacter spp. from other Enterobacter serotypes. In some embodiments, a sequence having at least 25 contiguous nucleotides of these sequences as well as complementary sequences and sequences comprising at least 90% nucleic acid sequence identity to SEQ ID NO:1, SEQ ID NO:2, SEQ ID NO:3, SEQ ID NO:4 SEQ ID NO:5, SEQ ID NO:6, SEQ ID NO:7, SEQ ID NO:8, SEQ ID NO:9, SEQ ID NO: 10, SEQ ID NO: 11 and SEQ ID NO:12, may also be used to identify and/or distinguish Cronobacter spp. from other Enterobacter serotypes.

Assays used for the detection and identification of Cronobacter spp. may include, but are not limited to, use of an oligonucleotide sequence of the disclosure for hybridization, and/or a primer pair that may be used for amplification (PCR) that may be designed based on the SEQ ID NOs: 1-12, and/or possibly in conjunction with a probe for real-time PCR. The length of an oligonucleotide probe and/or primer sequence may be as few as 10, at least 15, at least 20, at least 25, and up to 40 nucleotides in length. Use of larger than 40 nucleotide oligonucleotides are also contemplated. Design of sequences for hybridization detection and PCR may be done by one of skill in the art in light of the teachings of this disclosure, such as for example the unique sequences of Cronobacter spp.

### Example IV. Identification of Cronobacter spp. Specific and Unique Regions

To identify Cronobacter spp., the nine newly assembled draft Cronobacter genomes, the two published Cronobacter genomes, and 45 closely related Enterobacter genomes downloaded from NCBI were compared. An in-house pipeline called SIGA was used to align the genomes to a Cronobacter reference genome and identify sequence segments that are shared by all 11 Cronobacter genomes with at least 95% identity but are at least 20% divergent in all the other 45 Enterobacter genomes. The resulting sequences were screened against the GenBank bacterial, viral, fungal and plant sequences using BLASTN, and any sequence having a BLAST hit with at least 80% identity over 50 or more nucleotides was excluded from consideration. Cronobacter spp. specific signature sequences were obtained comprising 2,070 bp which are described in Table 3 and include sequences comprising SEQ ID NO:1, SEQ ID NO:2, SEQ ID NO:3, SEQ ID NO:4 SEQ ID NO:5, SEQ ID NO:6, SEQ ID NO:7, SEQ ID NO:8, SEQ ID NO:9, SEQ ID NO: 10, SEQ ID NO: 11 and SEQ ID NO:12.

### Example V. Identification of Species Specific and Unique Regions

The approach of Example V was also used to identify sequences that are present in available genomes of all strains (with at least 97% identity) of a particular Cronobacter species but are absent (at least 20% divergent) from any other Cronobacter or Enterobacter genomes. The analysis resulted in 102, 87, 69, 412, 135, 393, and 65 sequences longer than 100 nucleotides comprising 31, 18, 16, 111, 46, 100 and 28 kb that are specific to C. sakazakii, C. turicensis, C. malonaticus, C. muytjensii, C. genomosp1, C dublinensis, and C sakazakii 701 (a ST4 strain), respectively. Table 4 summarizes the number of signature sequences identified for each species. The sequences specific to each species are listed in SEQ ID NO: 16-117 (C. sakazakii), SEQ ID NO: 118-204 (C. turicensis), SEQ ID NO: 205-273 (C. malonaticus), SEQ ID NO: 274-685 (C. muytjensii), SEQ ID NO: 686-820 (C. genomosp1), SEQ ID NO: 821-1213 (C dublinensis) and SEQ ID NO: 1214-1278 (C sakazakii ST4 strain).

TABLE 4

| | | | Range of | | | |
|---|---|---|---|---|---|---|
| | Strains with | Num of | Sequence | | | |
| | available | Signature | Length | Average | Median | Total Length |
| Species | genomes | Sequences | (median) | Length (nt) | Length (nt) | (nt) |
| Cronobacter spp. | 680, 696, 701, | 12 | 104-448 | 173 | 148 | 2,070 |
| | BAA-894, 564, | | | | | |
| | z3032, 507, 681, | | | | | |
| | 530, 581, 582 | | | | | |
| C. sakazakii | 680, 696, 701, | 102 | 101-3,625 | 308 | 160 | 31,366 |
| | BAA-894 | | | | | |
| C. turicensis | 564, z3032 | 87 | 100-1,375 | 216 | 141 | 18,774 |
| C. malonaticus | 507, 681 | 69 | 101-2,265 | 234 | 160 | 16,162 |
| C. muytjensii | 530 | 412 | 100-3,966 | 270 | 153 | 111,143 |
| C. genomosp1 | 581 | 135 | 100-2,498 | 343 | 184 | 46,364 |
| C. dublinensis | 582 | 393 | 100-4,784 | 254 | 159 | 99,796 |
| C sakazakii ST4 | 701 | 65 | 100-3,556 | 429 | 250 | 27,858 |
| strain | | | | | | |

In some embodiments, the signature sequences, as well as complements and sequences comprising at least 90% nucleic acid sequence identity thereof can be used for designing genetic assays (real-time PCR, PCR) that specifically detect and differentiate Cronobacter spp. or particular Cronobacter species in samples such as but not limited to foods, beverages, clinical samples, environmental samples.

### Example VI. Assays to Specifically Detect Cronobacter Spp.

Exemplary real-time PCR assays were designed from specific and unique and specific Cronobacter spp. sequence regions SEQ ID NO:1, SEQ ID NO:2, SEQ ID NO:3, SEQ ID NO:4 SEQ ID NO:5, SEQ ID NO:6, SEQ ID NO:7, SEQ ID NO:8, SEQ ID NO:9, SEQ ID NO: 10, SEQ ID NO: 11 and SEQ ID NO:12. These identified Cronobacter spp. target sequences were used to design primers and probes for real-time PCR assays. Programs for assay design include Primer3 (Steve Rozen and Helen J. Skaletsky (2000) “Primer3” on the World Wide Web for general users and for biologist programmers as published in: Krawetz S, Misener S (eds) Bioinformatics Methods and Protocols: Methods in Molecular Biology. Humana Press, Totowa, N.J., pp 365-386), Primer Express® software (Applied Biosystems), and OLIGO 7 (Wojciech Rychlik (2007). “OLIGO 7 Primer Analysis Software”. Methods Mol. Biol. 402: 35-60)). The subsequently designed PCR primers and probes for use in assays by real-time PCR can detect unambiguously, specifically and with great sensitivity Cronobacter spp. The identified target sequences were used to design primers and probes for detection, identification, quantization, and/or differential detection of a Cronobacter spp. organism.

### Example VII. Assay to Specifically Detect Cronobacter Spp.

According to one embodiment, an exemplary method for detection of Cronobacter spp. may comprise hybridization a polynucleotide primer to a target nucleic acid sequence unique to Cronobacter spp; amplification of the target nucleic acid sequence using methods selected from the group comprising polymerase chain reaction (PCR), RT-PCR, asynchronous PCR (A-PCR), and asymmetric PCR (AM-PCR), strand displacement amplification (SDA), multiple displacement amplification (MDA), nucleic acid strand-based amplification (NASB A), rolling circle amplification (RCA), transcription-mediated amplification (TMA); and detection of the amplified target nucleic acid sequence is indicative of the presence of Cronobacter spp. in the test sample.

In one example embodiment a method may use a forward and reverse primer pair as described in SEQ ID NO: 13, SEQ ID NO: 14 (Table 5) for amplification as described above. Detection of amplified product may comprise using a probe set forth in SEQ ID NO: 15. Table 5 depicts primer pair and probe sequences that may be used in one example method for detecting Cronobacter spp. targeting the gene recN. This assay matches perfectly or near perfectly to all the nine Cronobacter draft genomes as well as the two public complete Cronobacter genomes, confirming its high coverage in detecting Cronobacter strains. Embodiments may use complements and labeled derivatives of the primer and probe sequences described.

TABLE 5

| Assay | | | |
|---|---|---|---|
| ID | Forward Primer | Reverse Primer | Probe |
| 120 | GAAGAGTACAAACGYTTAGCCAAYAG | CGGCCAGCAGGTTTAAYG | CTGCTGGCTGGTAGAAAG |
| | (SEQ ID NO: 13) | (SEQ ID NO: 14) | (SEQ ID NO: 15) |

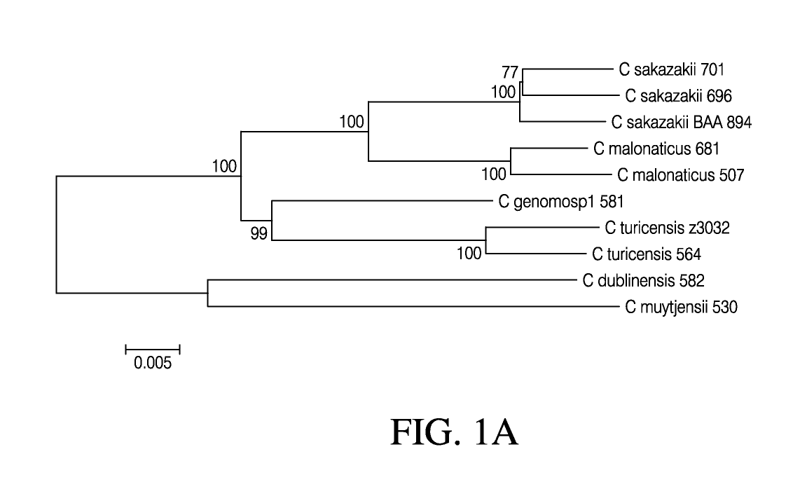

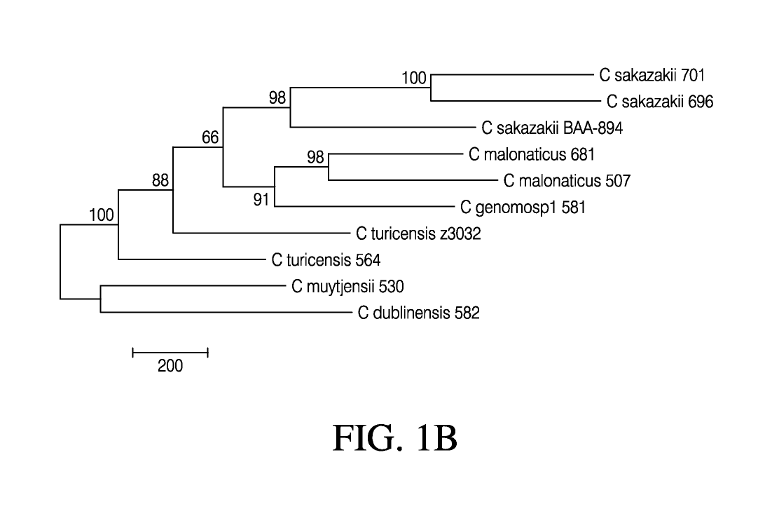

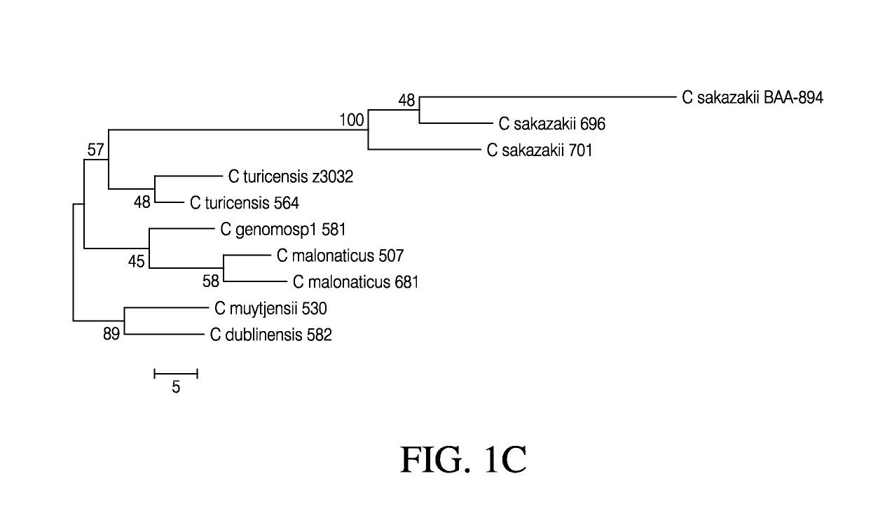

FIG. 1 is a phylogenetic analysis and shows the phylogenetic tree inferred on 100 core genes, the presence of genes from the pan-genome, and the presence of putative virulence genes. The values on the branches are bootstrap values based on 1,000 replicates. FIG. 1A is the neighbor joining tree inferred based on the concatenated DNA sequence alignment of 100 Cronobacter core genes (85,059 nt). FIG. 1B is maximum parsimony tree inferred based on the presence and absence of the 6,156 genes in the pan genome. FIG. 1C is maximum parsimony tree inferred based on the presence and absence of 174 putative virulence genes, including fimbrial clusters, iron uptake system, some C. sakazakii specific genes, and putative type VI seCretion system.

Table 6 depicts the presence of the C. sakazakii BAA-894 fimbrial clusters in the other nine strains.

TABLE 6

| C. sakazakii | | | | | | | | | | |
|---|---|---|---|---|---|---|---|---|---|---|
| BAA-894 Gene | Function | Csak701 | Csak696 | Cmal681 | Cmal507 | Csp1581 | Cturz3032 | Ctur564 | Cdub582 | Cmuy530 |
| ESA_01970- | Pilin FimA, Usher FimD, | − | − | − | − | − | − | − | − | − |
| ESA_01976 | Chaperone FimC | | | | | | | | | |
| ESA_02538 | Pilin FimA | + | + | + | + | + | + | − | − | + |
| ESA_02539- | Chaperone FimC, Usher | + | + | + | + | + | + | + | − | − |
| ESA_02541 | FimD, Pilin FimA (FimH) | | | | | | | | | |
| ESA_02542 | Putative minor component | + | + | − | − | − | + | + | − | − |
| | FimG | | | | | | | | | |
| ESA_02795 | Fimbrial protein | + | − | + | + | − | − | − | − | − |
| ESA_02796- | Pilin FimA, Usher FimD, | + | + | + | + | − | − | − | − | − |
| ESA_02798 | Chaperone FimC | | | | | | | | | |
| ESA_02799, | Putative fimbrial protein | + | + | − | − | − | − | − | − | − |
| ESA_04067, | | | | | | | | | | |
| ESA_04069 | | | | | | | | | | |
| ESA_04068 | Fimbrial protein | − | + | − | − | − | − | − | − | − |
| ESA_04070 | Fimbrial protein | + | + | + | + | + | + | + | − | − |
| ESA_04071 | Usher FimD | + | + | + | + | + | + | + | − | + |
| ESA_04072 | Chaperone FimC | + | + | + | + | + | + | + | + | + |
| ESA_04073 | Fimbrial protein | + | + | − | − | − | − | + | − | − |

Table 7 depicts the presence of the putative C. sakazakii BAA-894 iron uptake genes in the other nine strains.

TABLE 7

| C. sakazakii | | | | | | | | | | |
|---|---|---|---|---|---|---|---|---|---|---|
| BAA-894 Gene | Function | Csak701 | Csak696 | Cmal681 | Cmal507 | Csp1581 | Cturz3032 | Ctur564 | Cdub582 | Cmuy530 |
| ESA_00459 | fepE, ferric enterobactin | + | + | + | + | + | + | + | − | − |
| | transport | | | | | | | | | |
| ESA_00791 | fepC, hypothetical | + | + | + | + | + | + | + | + | + |
| | protein | | | | | | | | | |
| ESA_00792 | fepG, iron-enterobactin | − | + | + | + | + | + | + | − | + |
| | transporter permease | | | | | | | | | |
| ESA_00793 | fepD, iron-enterobactin | + | + | + | + | + | + | − | + | + |
| | transporter | | | | | | | | | |
| ESA_00794 | entS, enterobactin exporter | − | − | + | + | − | + | − | + | − |
| | EntS | | | | | | | | | |
| ESA_00796- | fepB, iron-enterobactin | + | + | + | + | + | + | + | + | + |
| ESA_00799 | transporter, entC, entB, | | | | | | | | | |
| | entA | | | | | | | | | |
| ESA_01552 | iroN, outer membrane | + | + | + | + | + | + | + | + | + |
| | receptor FepA | | | | | | | | | |
| ESA_02727 | entF, enterobactin synthase | + | + | + | + | + | + | + | + | + |
| | subunit F | | | | | | | | | |
| ESA_02729 | entE, enterobactin synthase | + | + | + | + | + | + | + | + | − |
| | subunit E | | | | | | | | | |
| ESA_02730 | fepA, outer membrane | + | + | + | + | + | + | + | + | + |
| | receptor FepA | | | | | | | | | |
| ESA_02731 | entD, hypothetical protein | + | + | + | + | + | + | + | + | + |
| ESA_03187 | fhuB, iron-hydroxamate | − | + | + | − | − | + | − | − | − |
| | transporter permease | | | | | | | | | |
| ESA_03188 | fhuD, iron-hydroxamate | + | + | + | + | + | + | + | + | + |
| | transporter | | | | | | | | | |
| ESA_03190 | fhuA, ferrichrome outer | + | + | + | + | + | + | + | + | + |
| | membrane transporter | | | | | | | | | |
| ESA_03959 | ibpB, heat shock chaperone | + | + | + | + | + | + | + | + | + |
| | lbpB | | | | | | | | | |
| ESA_03960 | ibpA, heat shock | + | + | + | + | + | + | + | + | + |
| | protein lbpA | | | | | | | | | |
| pESA3p05547 | iucA, hypothetical protein | − | + | + | + | + | + | + | − | + |
| pESA3p05548- | iucB, iucC, iucD | + | + | + | + | + | + | + | + | + |
| pESA3p05550 | | | | | | | | | | |
| pESA3p05551 | iutA, hypothetical protein | + | + | + | + | − | + | + | + | + |

While the foregoing specification teaches the principles of the present claimed embodiments, with examples provided for the purpose of illustration, it will be appreciated by one skilled in the art from reading this disclosure that various changes in form and detail can be made without departing from the spirit and scope of the invention. These methods are not limited to any particular type of nucleic acid sample: plant, bacterial, animal (including human) total genome DNA, RNA, cDNA and the like may be analyzed using some or all of the methods disclosed in this invention. This invention provides a powerful tool for analysis of complex nucleic acid samples. From experiment design to detection of Cronobacter spp. assay results, the above invention provides for fast, efficient and inexpensive methods for detection of pathogenic Cronobacter spp.

### Example VIII. Control Based Threshold (CBT) Variation

The present example describes testing an example embodiment of a computer program of the disclosure with instructions being executed on a processor so as to perform a method for PCR analysis.

In one embodiment, as described in sections above, a control based threshold (CBT) method may comprise providing CBT instructions to a user for setting a threshold. Instructions to a user may indicate that a user set a threshold line at the level of a positive control plateau and then lower it to a pre-defined percentage of the plateau.

To test how much the Ct's would change if the threshold was set artificially to a “high” CBT (above the control threshold) and/or to a “low” CBT (below the control threshold), data was analyzed from almost 200 field samples (having approximately 100 positive and 100 negative samples) on three runs.

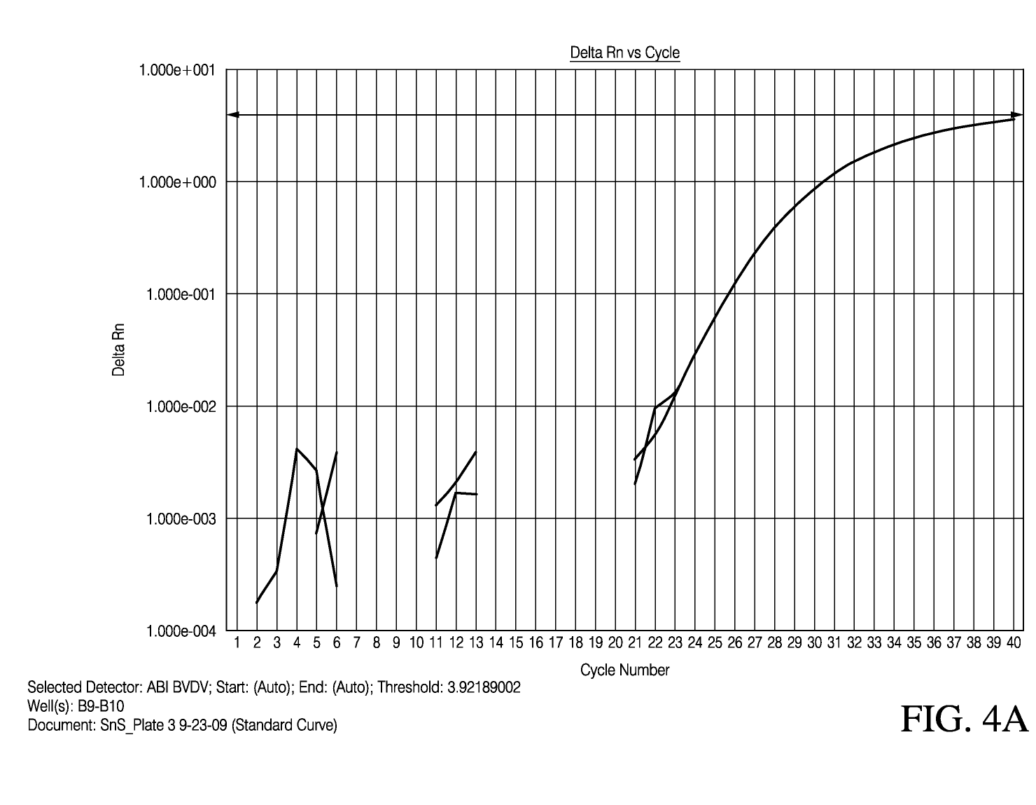

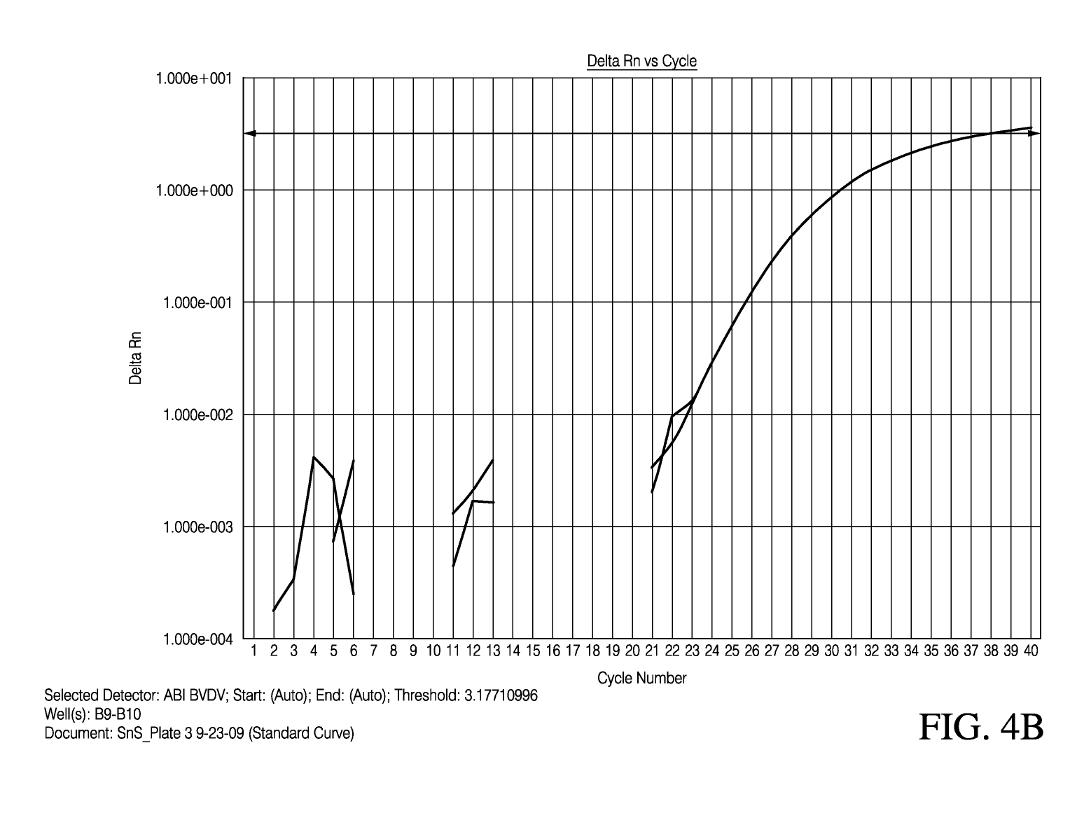

The results are shown in FIG. 4A for artificially set “high” CBT (above the control threshold) and in FIG. 4B for artificially set “low” CBT (below the control threshold) in comparison to CBT set by methods of the disclosure, and show that there were no changes in diagnostic calls (see FIGS. 4A and 4B).

### Example IX. CBT Variation

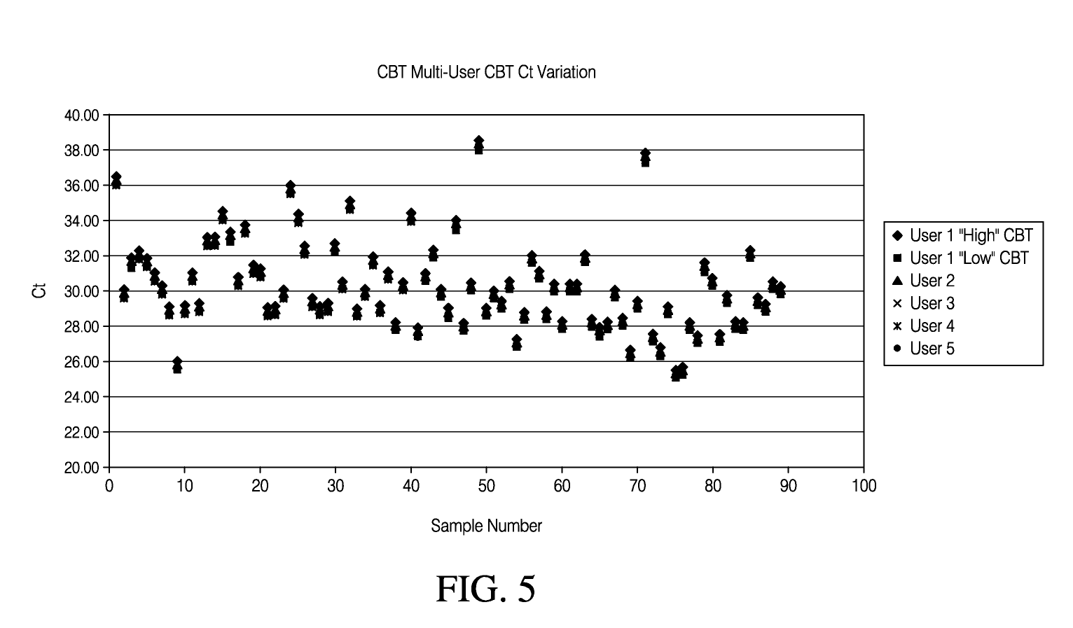

In this experiment, five different users were asked to analyze data from a PCR run using the Control-Based Threshold method. There was minimal variation between users in threshold setting and diagnostic calls were consistent between all 5 users. Results of this experiment are shown in FIG. 5, which show that setting a threshold using the present CBT method and algorithms provide consistent analysis of PCR data between different users.

### Example X. CBT Threshold Procedure

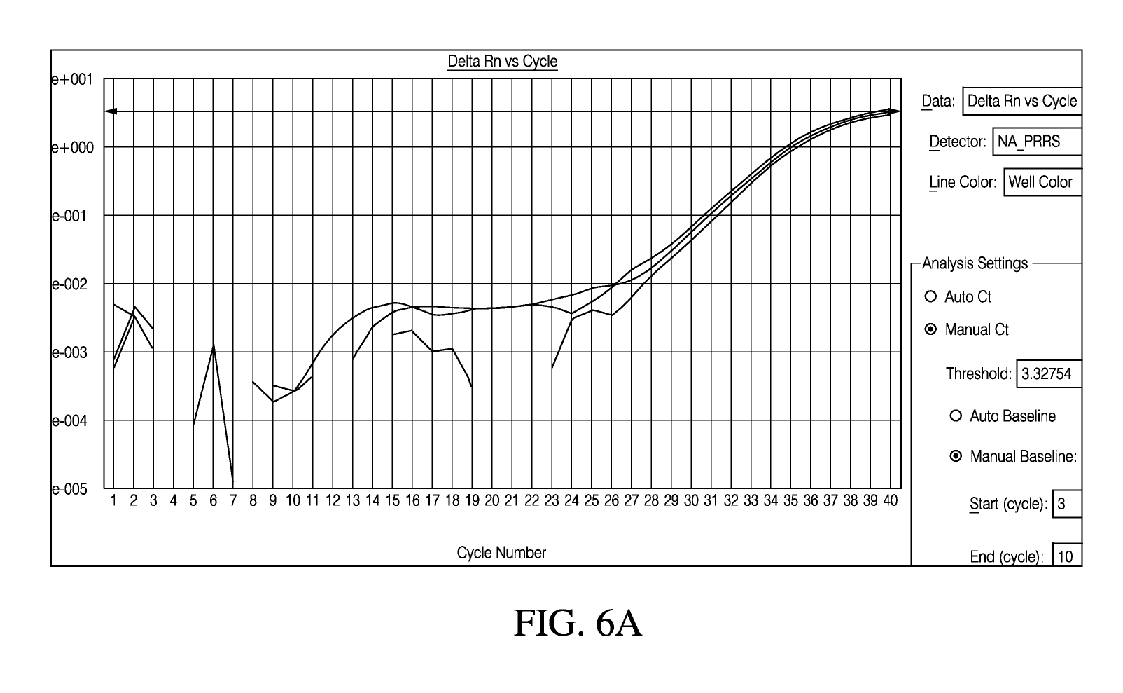

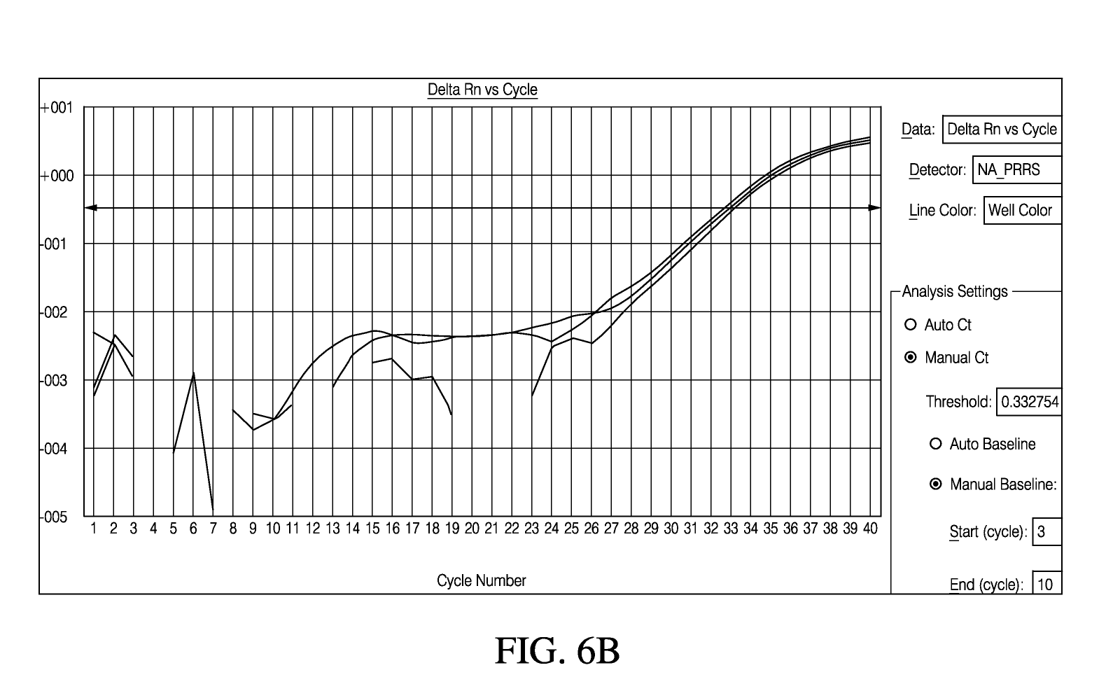

A graphical representation of one example manual way to obtain the proper threshold using CBT is shown in FIGS. 6A and 6B. Such a method may comprise determining which wells contain a positive PCR control; determining the dRN of the positive control at cycle 40 (or the last PCR cycle number chosen by user) (for example, this may be by dragging the threshold up to the plateau and reading the result of exporting the dRN into an excel spreadsheet and calculating the average dRN at cycle 40 of the positive controls); taking the pre-defined % of the plateau for that assay (the CBT) and calculating the threshold; the threshold value may then be typed into software (or GUI).

FIGS. 6A and 6B depict results of a CBT threshold procedure and show Step 1 comprising position threshold at plateau of positive control and record instrument dRn value which equals 3.32754 (FIG. 6A) and Step 2 shows position threshold at a pre-defined % of plateau of positive control determined in step 1 which is equal to 0.332754 (FIG. 6B).

### Example XI. Utilization of CBT Threshold

Threshold settings Allows for reproducible results across multiple users and multiple laboratories. Minimal variation observed in Ct values (<1 Ct) among users. No changes in positive or negative calls between users.

Deviation from user provided instructions: Example of the variation in threshold setting needed to change the results by 2Ct's; Significantly greater that what is seen with CBT method users; and Example below would be considered off-label use of product

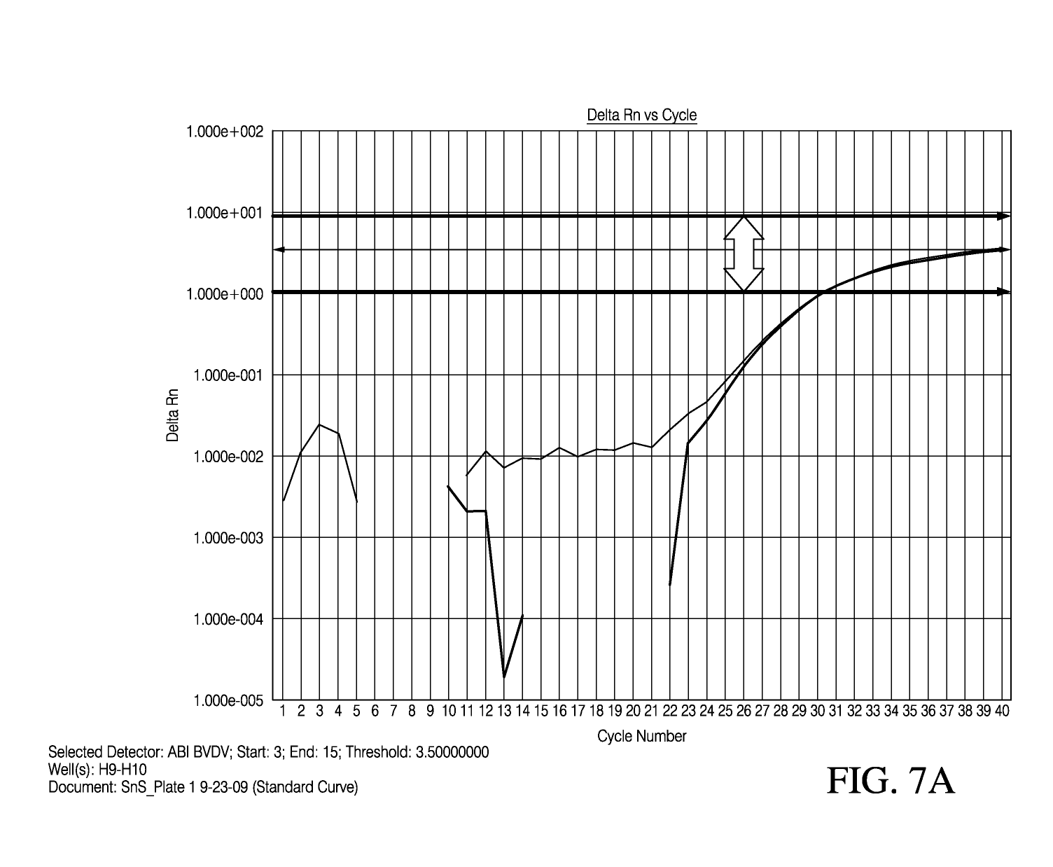

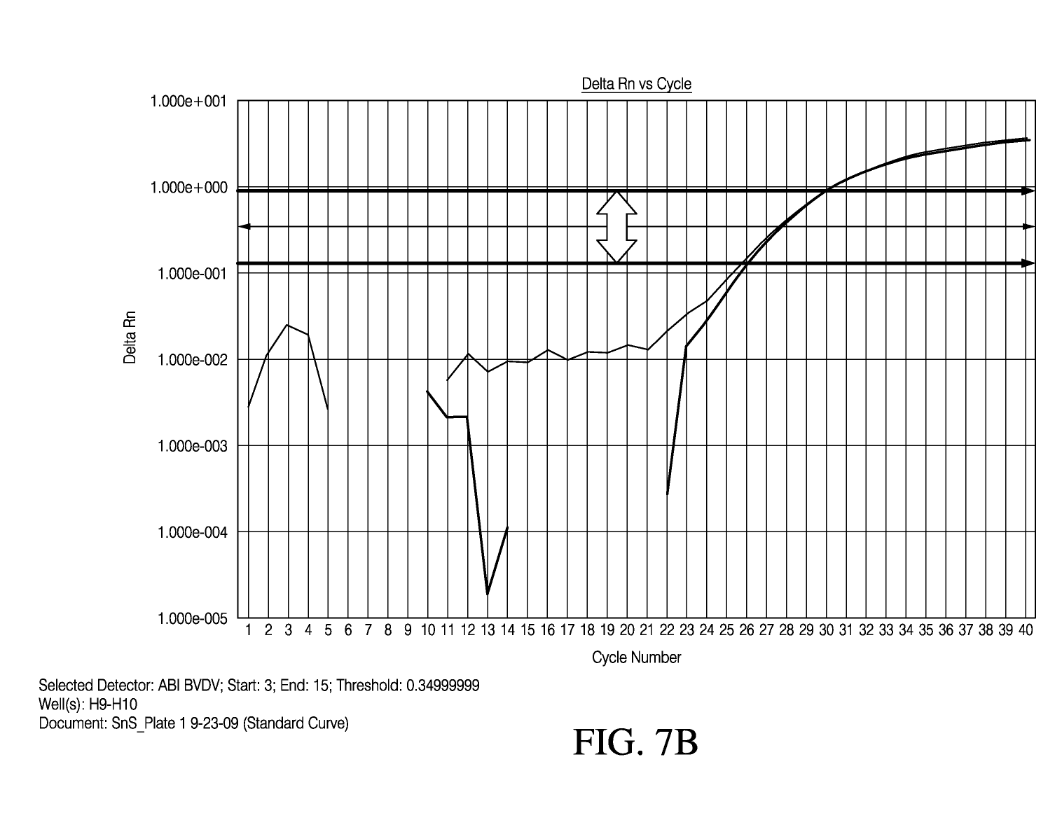

FIG. 7A and FIG. 7B depict that the present CBT is more consistent than other threshold setting methods. As shown above, FIG. 4 showed high level of consistency between multiple users employing the CBT method. For most of the assays tested by the present inventors have CT ranges in which the sample is called as “suspect”. For example, if a sample has a CT between 38-40CT's it is considered a suspect sample. To address if a user may be able to arrive at desired results by making small adjustments to the threshold tests were performed that showed that even relatively large changes to the threshold setting do not result in changes in sample calls from positive to negative. It was shown that to change a call from a positive a user would have to adjust the threshold so much that it would result in a at least 2CT difference. FIGS. 6A and 6B show graphs showing the amount of variation in threshold setting that would be need to change the CT by >2. This is significantly more that what is shown in FIG. 3A or 4.

Ability to modify threshold setting to influence result:
CBT is based on set % from the assay control positive amplification plot. Data above confirm that the amount of CBT variation between upper and lower settings is less than 1 Ct.
No change in positive or negative calls in experiments with 5 separate users (When following instructions for use, cannot change a diagnostic call).
Additional Control—Suspect Workflow in 38-40 Ct range: Even if threshold is adjusted to maximum for a borderline result, 37Ct for example, the result would be pushed into 38-40 “suspect workflow” that recommends additional testing and confirmation by another method if inconclusive.
Deviating from the method (% range) would be is an off-label use of the product and would not be supported.
Real-Time PCR data (.sds files) clearly display what % range was used to make a diagnostic call.

All publications and patent applications cited herein are incorporated by reference in their entirety for all purposes to the same extent as if each individual publication or patent application were specifically and individually indicated to be so incorporated by reference. Although the present invention has been described in some detail by way of illustration and example for purposes of clarity and understanding, it will be apparent that certain changes and modifications may be practiced within the scope of the appended claims.

### REFERENCES

The following references, to the extent that they provide exemplary procedural or other details supplementary to those set forth herein, are specifically incorporated herein by reference:
1: Aziz, R. K., et al. (2008) The RAST Server: rapid annotations using subsystems technology, BMC Genomics, 9, 75.
2: Joseph, S, and Forsythe, S. (2011) Predominance of Cronobacter sakazakii ST4 in neonatal infections. Emerging Infectious Disease, In Press.
3: Kucerova, E., et al. (2010) Genome sequence of Cronobacter sakazakii BAA-894 and comparative genomic hybridization analysis with other Cronobacter species, PLoS One, 5, e9556.
4: Kucerova, E., Joseph, S, and Forsythe, S. (2011) Cronobacter: ubiquity and diversity. Quality Assurance and Safety of Crops and Foods, In Press.
5: Kurtz, S., et al. (2004) Versatile and open software for comparing large genomes, Genome Biol, 5, R12.
6: Stephan, R., et al. (2011) Complete genome sequence of Cronobacter turicensis LMG 23827, a food-borne pathogen causing deaths in neonates, J Bacteriol, 193, 309-310. Zerbino, D. R. and Birney, E. (2008) Velvet: algorithms for de novo short read assembly using de Bruijn graphs, Genome Res, 18, 821-829.
7: Lai, K. K. (2001) Enterobacter sakazakii infections among neonates, infants, children, and adults. Case reports and a review of the literature, Medicine (Baltimore), 80, 113-122.

1. An isolated nucleic acid sequence having SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO: 10, SEQ ID NO: 11, SEQ ID NO:12, fragments thereof, at least 25 contiguous nucleotide sequences thereof, complements thereof or sequences comprising at least 90% nucleic acid sequence identity thereto.

2-13. (canceled)

14. An isolated nucleic acid sequence comprising SEQ ID NO: 13-1278, complements thereof, fragments thereof and labeled derivatives thereof and sequences comprising at least 90% nucleic acid sequence identity thereof.

15. (canceled)

16. A method of distinguishing an organism belonging to a Cronobacter spp. from a non-Cronobacter spp. strain comprising: detecting at least one nucleic acid sequence comprising SEQ ID NO:1, SEQ ID NO:2, SEQ ID NO:3, SEQ ID NO:4 SEQ ID NO:5, SEQ ID NO:6, SEQ ID NO:7, SEQ ID NO:8, SEQ ID NO:9, SEQ ID NO: 10, SEQ ID NO: 11, SEQ ID NO:12, fragments thereof or complements thereof, wherein detection of one of these nucleic acid sequences is indicative of the presence of a Cronobacter spp and the absence of a non-Cronobacter spp.

17. The method of claim 16, wherein detecting the at least one nucleic acid sequence comprises at least one technology selected from the group consisting of amplification, hybridization, mass spectrometry, nanostring, microfluidics, chemiluminescence, enzyme technologies and combinations thereof.

18. The method of claim 17, wherein amplification is selected from the group consisting of polymerase chain reaction (PCR), RT-PCR, asynchronous PCR (A-PCR), and asymmetric PCR (AM-PCR), strand displacement amplification (SDA), multiple displacement amplification (MDA), nucleic acid strand-based amplification (NASBA), rolling circle amplification (RCA), transcription-mediated amplification (TMA).

19. The method of claim 16, further comprising isolating nucleic acid from a sample suspected of being contaminated with a Cronobacter organism.

20. The method of claim 19, wherein the sample is a food sample, an agricultural sample, a produce sample, an animal sample, an environmental sample, a biological sample, a water sample and an air sample.

21. A method for detecting Cronobacter spp. in a sample comprising the steps of:
a) providing an isolated nucleotide sequence of a Cronobacter spp. specific nucleotide sequence comprising SEQ ID NO:1, SEQ ID NO:2, SEQ ID NO:3, SEQ ID NO:4 SEQ ID NO:5, SEQ ID NO:6, SEQ ID NO:7, SEQ ID NO:8, SEQ ID NO:9, SEQ ID NO: 10, SEQ ID NO: 11, SEQ ID NO:12, fragments thereof, at least 25 nucleotide sequences thereof, complements thereof, sequences comprising at least 90% nucleic acid sequence identity thereof, or a labeled derivative thereof;
b) contacting the isolated nucleotide sequence with the sample under hybridization conditions; and
c) detecting hybridization of at least one of the isolated nucleotide sequences of a Cronobacter spp. specific nucleotide sequence to a complementary nucleotide sequence in the sample,
wherein detection of a hybrid molecule is indicative of the presence of a Cronobacter spp in the sample.

22. A method for detecting a Cronobacter spp. in a sample comprising the steps of:
a) hybridizing at least a first pair of polynucleotide primers to a first target nucleic acid sequence specific to the Cronobacter spp.;
b) amplifying the first target nucleic acid sequence or a fragment thereof to form a first amplified target nucleic acid sequence product; and
c) detecting the at least first amplified target nucleic acid sequence product, wherein detection of the at least first amplified target nucleic acid sequence product is indicative of the presence of the Cronobacter spp. in the sample.

23. The method of claim 22 further comprising:
a) hybridizing a second pair of polynucleotide primers to the second target nucleic acid sequence specific to the Cronobacter spp.;
b) amplifying the second target nucleic acid sequence to form a second amplified target nucleic acid sequence product; and
c) detecting the second amplified target nucleic acid sequence product, wherein detection of the second amplified target nucleic acid sequence product is indicative of the presence of the Cronobacter spp.

24. The method of claim 23 wherein the first target nucleic acid sequence specific to Cronobacter spp. and the second target nucleic acid sequence specific to Cronobacter spp. are selected from the group consisting of SEQ ID NO:1, SEQ ID NO:2, SEQ ID NO:3, SEQ ID NO:4 SEQ ID NO:5, SEQ ID NO:6, SEQ ID NO:7, SEQ ID NO:8, SEQ ID NO:9, SEQ ID NO: 10, SEQ ID NO: 11, SEQ ID NO:12, fragments thereof, at least 25 nucleotide sequences thereof, complements thereof and sequences comprising at least 90% nucleic acid sequence identity thereof, wherein the first and the second target nucleic acids are different from each other.

25. The method of claim 24 wherein the first or the second primer pair comprises SEQ ID NO:13 and SEQ ID NO:14 complements thereof, and labeled derivatives thereof.

26. The method of claim 25, wherein the detecting comprises using a probe having SEQ ID NO:15 complements thereof, and labeled derivatives thereof.

27. The method of claim 22 further comprising detecting the species of the Cronobacter comprising:
detecting a Cronobacter species-specific target nucleic acid sequence comprising detecting the presence of at least one nucleic acid selected from SEQ ID NOs:16-1278, wherein the detection of a nucleic acid having SEQ ID NO: 16-117 is indicative of the presence of C. sakazakii, the detection of a nucleic acid having SEQ ID NOs:118-204 is indicative of the presence of C. turicensis, the detection of a nucleic acid having SEQ ID NOs:205-273 is indicative of the presence of C. malonaticus; the detection of a nucleic acid having SEQ ID NOs:274-685 is indicative of the presence of C. muytjensii, the detection of a nucleic acid having SEQ ID NOs:686-820 is indicative of the presence of C. dublinensis; the detection of a nucleic acid having SEQ ID NOs:821-1213 is indicative of the presence of C. genomosp. 1; and the detection of a nucleic acid having SEQ ID NOs:1214-1278 is indicative of the presence of C. sakazakii ST4 strain.

28. A method for distinguishing an organism from an Cronobacter spp. comprising analyzing the genome of the organism for the presence of a sequence selected from the group consisting of SEQ ID NO:1, SEQ ID NO:2, SEQ ID NO:3, SEQ ID NO:4 SEQ ID NO:5, SEQ ID NO:6, SEQ ID NO:7, SEQ ID NO:8, SEQ ID NO:9, SEQ ID NO: 10, SEQ ID NO: 11, SEQ ID NO:12, fragments thereof, at least 25 nucleotide sequences thereof and sequences comprising at least 90% nucleic acid sequence identity thereof by the method of claim 1.

29. The method of claim 28, wherein the organism is a strain of Enterobacter.

30. A kit for the detection of Cronobacter spp. comprising:
at least one pair of PCR primers designed to bind to and hybridize to at least one or more nucleic acid sequences of SEQ ID NO:1, SEQ ID NO:2, SEQ ID NO:3, SEQ ID NO:4 SEQ ID NO:5, SEQ ID NO:6, SEQ ID NO:7, SEQ ID NO:8, SEQ ID NO:9, SEQ ID NO: 10, SEQ ID NO: 11, SEQ ID NO:12, fragments thereof, complementary sequences thereof, sequences comprising at least 90% nucleic acid sequence identity thereof and complementary sequences comprising at least 90% nucleic acid sequence identity thereof; and
at least one probe designed to bind to and hybridize to a PCR product formed by amplification of the nucleic acid sequences of SEQ ID NO:1, SEQ ID NO:2, SEQ ID NO:3, SEQ ID NO:4 SEQ ID NO:5, SEQ ID NO:6, SEQ ID NO:7, SEQ ID NO:8, SEQ ID NO:9, SEQ ID NO: 10, SEQ ID NO: 11, SEQ ID NO:12, fragments thereof, complementary sequences thereof, sequences comprising at least 90% nucleic acid sequence identity thereof and complementary sequences comprising at least 90% nucleic acid sequence identity thereof.

31. The kit of claim 30, further comprising one or more components selected from a group consisting of: at least one enzyme, dNTPs, at least one buffer, at least one salt, at least one control nucleic acid sample and an instruction protocol.

32. The kit of claim 30, wherein the probe is labeled.

33. The kit of claim 30, wherein at least one primers of the PCR primer pair is a labeled primer.

34. The kit of claim 28, wherein:
at least one pair of PCR primer selected from a group of nucleic acid sequences comprising of SEQ ID NO:13, SEQ ID NO:14, SEQ ID NO:15, fragments comprising at least 10 contiguous nucleotide sequences thereof, complements thereof and labeled derivatives thereof; and
at least one probe selected from a group of nucleic acid sequences consisting of SEQ ID NO:13, SEQ ID NO:14, SEQ ID NO:15, fragments comprising at least 10 contiguous nucleotide sequences thereof, complements thereof and labeled derivatives thereof.

35. A kit for the detection of Cronobacter spp. comprising:
at least one pair of PCR primers designed to bind to and hybridize to at least one or more nucleic acid sequences of SEQ ID NOs:1-12, SEQ ID NOs:16-1278, fragments thereof, complementary sequences thereof, sequences comprising at least 90% nucleic acid sequence identity thereof and complementary sequences comprising at least 90% nucleic acid sequence identity thereof; and
at least one probe designed to bind to and hybridize to a PCR product formed by amplification of the nucleic acid sequences of SEQ ID NOs:1-12, SEQ ID NOs:16-1278, fragments thereof, complementary sequences thereof, sequences comprising at least 90% nucleic acid sequence identity thereof and complementary sequences comprising at least 90% nucleic acid sequence identity thereof; and
one or more components selected from a group consisting of: at least one enzyme, dNTPs, at least one buffer, at least one salt, at least one control nucleic acid sample and an instruction protocol.

36. A method for detecting a Cronobacter species or strain in a sample comprising:
a) hybridizing a first pair of polynucleotide primers to the at least a first target nucleic acid sequence specific to the first Cronobacter species or strain;
b) amplifying the at least first target nucleic acid sequence specific to the first Cronobacter species or strain to form a first amplified target nucleic acid sequence product; and
c) detecting the first amplified target nucleic acid sequence product, wherein detection of the first amplified target nucleic acid sequence product is indicative of the presence of the first Cronobacter species or strain.

37. The method of claim 36 further operable to distinguish between Cronobacter species or strain of the species C. sakazakii, C. turicensis, C. malonaticus, C. muytjensii, C. dublinensis, C. genomosp. 1 and C. sakazakii ST4 strain.

38. The method of claim 37 wherein the at least first target nucleic acids may comprise isolated sequences described in SEQ ID NOs:16-1278, fragments thereof, at least 25 nucleotide sequences thereof, complements thereof and sequences comprising at least 90% nucleic acid sequence identity thereof.

39-41. (canceled)
